(19) United States
(12) Patent Application Publication
(10) Pub. No.: US 2003/0207271 A1
(43) Pub. Date: Nov. 6, 2003

(54) Methods and compositions for biological sensors

(76) Inventors: Eric A. Holwitt, San Antonio, TX (US); Johnathan L. Kiel, Universal City, TX (US)

Correspondence Address: Blakely, Sokoloff, Taylor & Zafman, Seventh Floor, 12400 Wilshire Boulevard, Los Angeles, CA, 90025-1030, US

(21) Appl. No.: 10/034,127

(22) Filed: Dec. 27, 2001

Related U.S. Application Data
(63) Continuation-in-part of application No. 09/608,706, filed on Jun. 30, 2000, now Pat. No. 6,303,316.
(60) Provisional application No. 60/258,518, filed on Dec. 28, 2000.

(51) Int. Cl. C12Q 1/68; C12N 15/74; C12N 1/21

(52) U.S. Cl. 435/6; 435/455; 435/488; 435/252.33

(57) ABSTRACT

The present invention concerns compositions, apparatus and methods of use of recognition complexes, comprising biological sensors operably linked to an organic semiconductor. Multiple recognition complexes can be associated into a recognition complex system. The recognition complex system is of use to identify analytes, to separate biological sensors that bind to a target analyte from those that do not, to separate analytes that bind to a specific biological sensor from those that do not, and to prepare biological sensors with a high affinity for a particular analyte. The recognition complex system may be attached to a variety of surfaces, such as a chip, a flow cell, magnetic beads or non-magnetic beads. The biological sensor may be used for screening of, for example, a phage library, combinatorial chemistry library, plant tissue extract or animal tissue extract for inhibitors, activators or binding factors of bioactive molecules.

This application claims the benefit under 35 U.S.C. §119(e) of provisional U.S. patent application serial No. 60/258,518, filed on Dec. 28, 2000. This application is a continuation-in-part of U.S. patent application Ser. No. 09/608,706, filed Jun. 30, 2000 (now issued U.S. Pat. No. 6,303,316).

The invention described herein was made with Government support under contracts F41622-96-D-008 and F41824-00-D-700 awarded by the Department of the Air Force and Department of Energy contract number DE-AC06-76RL01830. The Federal Government has a nonexclusive, nontransferable, irrevocable, paid-up license to practice or have practiced for or on behalf of the United States the subject invention.

### BACKGROUND OF THE INVENTION

1. Field of the Invention

The present invention relates to the field of detection and identification of analytes, using novel compositions and apparatus comprising one or more biological sensors operably coupled to an organic semiconductor. More particularly the present invention relates to novel methods for preparing and identifying biological sensors that can bind to various analyte molecules, such as proteins, peptides, transcription factors, enzymes, receptors, antibodies or hormones. Other aspects of the present invention concern compositions and methods of use of organic semiconductors attached to biological sensors for detecting and/or identifying various analytes. The present invention further relates to the detection, identification and neutralization of chemical and biological warfare agents.

2. Description of Related Art

There is a great need for the development of methods and apparatus capable of detecting and identifying known or unknown chemical and biological agents (herein referred to as analytes), which include but are not limited to nucleic acids, proteins, illicit drugs, explosives, toxins, pharmaceuticals, carcinogens, poisons, allergens, contaminants, pathogens and infectious agents. Possible approaches to this problem include the use of nucleic acid microchip technology, flow cytometry, flow cell technology or magnetic bead technology. Although these technologies are known for various applications, the present invention provides a novel and unexpected use of these technologies to detect and identify known or unknown analytes.

As one skilled in the art will readily appreciate, any method, technique or device capable of such detection and identification would have numerous medical, industrial forensic and military applications. For instance, such methods, techniques and devices could be employed in the diagnosis and treatment of disease, to develop new compounds for pharmaceutical, medical or industrial purposes, or to identify chemical and biological warfare agents.

Current methods, techniques and devices that have been applied to identification of chemical and biological analytes typically involve capturing the analyte through the use of a non-specific solid surface or through capture deoxyribonucleic acids (DNA) or antibodies. A number of known binding agents must then be applied, particularly in the case of biological analytes, until a binding agent with a high degree of affinity for the analyte is identified. A labeled antiligand (e.g., labeled DNA or labeled antibodies) must be applied, where the antiligand causes, for example, the color or fluorescence of the analyte to change if the binding agent exhibits affinity for the analyte (i.e., the binding agent binds with the analyte). The analyte may be identified by studying which of the various binding agents exhibited the greatest degree of affinity for the analyte.

There are a number of problems associated with current methods of chemical and biological agent identification. It takes a great deal of time and effort to repetitiously apply each of the known labeled antiligands, until an antiligand exhibiting a high degree of affinity is found. Accordingly, these techniques are not conducive to easy automation. Current methods are also not sufficiently robust to work in the heat, dust, humidity or other environmental conditions that might be encountered, for example, on a battlefield or in a food processing plant. Portability and ease of use are also problems seen with current methods for chemical and biological agent identification.

There is also a need in the art for methods of high through-put screening for compounds that can activate, inhibit or bind to various biologically active molecules, such as enzymes, receptor proteins, cytokines, hormones, growth factors, cell adhesion factors, angiogenic agents, nucleic acids and lipids. Such methods may be of use, for example, to identify or characterize new pharmaceutical or therapeutic agents, or to identify the active component(s) in a complex mixture of compounds such as a cell or tissue extract. Screening of native plant extracts for active components is a non-limiting example of such a use. Present methods for high through-put screening with protein or peptide ligands tend to have the same problems of stability, portability and ease of automation seen with methods of chemical and biological agent identification, discussed above. Biological sensors that have been selected to bind to known activators or inhibitors of biologically active molecules may be used to screen for previously unknown analogs of such activators or inhibitors.

### SUMMARY OF THE INVENTION

The present invention fulfills an unresolved need in the art, by providing compositions and methods of production and use of biological sensors that are capable of detecting, identifying, characterizing or purifying a chemical or biological agent (hereafter, “analyte”), preparing or purifying high affinity biological sensors for selected known analytes, using high affinity biological sensors to measure the concentration of analyte in a sample or to neutralize an analyte, or to perform high through-put screening of libraries of compounds or native plant extracts for compounds that are structural analogs of known inhibitors, activators or binding agents of bioactive molecules.

In some embodiments, the biological sensors may be produced and screened by incorporation into regulatory regions of genes, such as promotor sequences. In preferred embodiments, the biological sensor and associated gene may be incorporated into a bacterium, virus, eukaryotic cell or other expression system. The biological sensor incorporated into the regulatory region may bind to an analyte within the expression system. Binding of biological sensor to a target analyte results in a detectable change in gene expression, allowing the selection of bacteria, viruses or cells that contain a biological sensor sequence with affinity for the analyte.

Certain embodiments concern compositions comprising biological sensors, such as recognition complexes. Each recognition complex is comprised of a biological sensor operably coupled to an organic semiconductor. In preferred embodiments, the organic semiconductor is DAT (polydiazoaminotyrosine) or DALM (diazoluminomelanin), although the use of other organic semiconductors is contemplated within the scope of the invention. In various embodiments, the organic semiconductor may be attached to the biological sensor by either covalent or non-covalent interaction.

In preferred embodiments, the biological sensor is DNA, although it is contemplated that other nucleic acids comprised of RNA or synthetic nucleotide analogs could be utilized as well. In certain embodiments, the biological sensor sequences are random, or may be generated from libraries of random DNA sequences. In other embodiments, the biological sensor sequences may not be random, but may rather be designed to react with specific target analytes. In certain properties, such as their ability to bind to proteins, peptides and other analytes, the biological sensor sequences resemble aptamers (Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823 and PCT application WO 99/31275).

In certain embodiments, the analyte to be identified may be added in the form of a complex mixture that may include, for example, aqueous or organic solvent, proteins, lipids, nucleic acids, detergents, particulates, intact cells, bacteria, viruses and spores, as well as other components. In other embodiments, the analyte may be partially or fully purified before exposure to the array.

In certain embodiments, a recognition complex system, comprising two or more recognition complexes, may be used in methods for identifying an analyte. After the analyte is contacted with the recognition complexes, certain recognition complexes will bind the analyte, while others will not. Binding of analyte to a recognition complex may be detected by changes in the photochemical properties of the biological sensor/organic semiconductor couplet upon binding to the analyte. Nonlimiting examples of photochemical signals include fluorescent, phosphorescent and luminescent signals as well as changes in color. The degree to which the photochemical properties change is a function of the degree to which the biological sensor binds the analyte. Accordingly, the photochemical changes that occur across all of the recognition complexes, when taken as a whole, can be used as a unique signature to identify the analyte. To facilitate detection of such photochemical changes, the recognition complex system may be associated with a detection unit operably coupled to the recognition complexes. Non-limiting examples of detection units include a charge coupled device (CCD), a CCD camera, a photomultiplier tube, a spectrophotometer or a fluorometer. The recognition complex system may also be associated with system memory for storing photochemical signals, as well as a data processing unit that may comprise a neural network or lookup tables.

In addition to analyte identification, recognition complexes may be used to screen for the presence or measure the amount of analytes that are biological molecules, such as hormones, cytokines, vitamins, metabolites or other compounds, in samples of human tissue, fluids or extracts. Biological sensors with high affinity for specific target molecules of interest may be prepared as described below. Upon exposure of recognition complexes incorporating the high affinity biological sensors to a sample, the presence of the target molecule is indicated by its binding to the biological sensor. Since binding of analyte to biological sensor results in a photochemical signal, the concentration of analyte in the sample can be readily determined by quantifying the signal. Where the analyte of interest is part of a macromolecular complex, flow cytometry may also be used to detect and quantify the amount of analyte in a sample.

In certain embodiments, the recognition complex system may be used to enrich or purify analytes that bind to one or more selected biological sensors. In a preferred embodiment, selected biological sensors are attached to a surface and exposed to a population of analytes. After binding of analyte to biological sensor, the unbound analytes are removed and the enriched or purified bound analyte is eluted from the biological sensor. Enrichment and purification may occur using either an iterative process, with multiple cycles of binding, separation and elution, or by a single-step process. Separation of bound from unbound analyte may occur by any method known in the art. In a non-limiting example, the biological sensors may be attached to a column chromatography resin or other solid support and exposed to a mixture of analytes. Unbound analyte may be removed by simple washing of the column or other support. Bound analyte may be eluted by exposure to solutions containing appropriate salt concentration, pH, detergent content, chaotrophic agent or other substance that interferes with the binding interaction. Alternatively, bound analyte may be eluted by heating the solution. Depending on the affinity of analyte for biological sensor and the stringency of the initial binding interaction, it may be possible to obtain a relatively purified analyte with a single binding step.

Other embodiments of the present invention concern methods of use of a recognition complex system for producing information regarding specific chemical and biological properties of an unknown analyte. For example, complex mixtures containing analytes may be screened for binding to one or more biological sensors that have high affinity for a known activator, inhibitor or binding factor of a bioactive molecule, such as a specific enzyme, receptor protein, transport protein, binding protein, cytokine, transcription factor, protein kinase, structural protein, hormone, growth factor, cell adhesion factor, angiogenic agent, nucleic acid or lipid. In these embodiments, the biological sensor acts as a substitute for the bioactive molecule and is used to screen complex mixtures for structural analogs of the inhibitor, activator or binding factor. Such substitution may be desirable, for example, where the bioactive molecule is unstable or difficult to obtain in purified form. Unknown analytes that bind to the biological sensor are identified as putative inhibitors, activators or binding factors for the target bioactive molecule, since they share enough structural homology bind to the same biological sensor as a known inhibitor, activator or binding factor.

Analytes that bind to the biological sensor may be enriched or purified as discussed above. The identity of the purified analyte as a novel inhibitor, activator or binding factor of the bioactive molecule may be confirmed by standard methods known in the art for characterizing inhibitors, activators or binding factors.

In preferred embodiments, the analytes used in the above methods may include random amino acid sequences. A non-limiting example of such sequences would consist of a phage display library (see U.S. Pat. Nos. 5,565,332, 5,596,079, 6,031,071 and 6,068,829, incorporated herein by reference in their entirety.) In other preferred embodiments, the analytes may consist of combinatorial chemical libraries (as a non-limiting example, U.S. Pat. No. 5,565,324, incorporated by reference in its entirety). In other preferred embodiments, the complex mixtures to be screened may consist of extracts of plant or animal tissues or cell culture lines.

In certain embodiments, the recognition complexes may be attached to a surface, such as a Langmuir-Blodgett film, functionalized glass, germanium, silicon, PTFE, polystyrene, gallium arsenide, gold, silver, membrane, nylon, glass bead, magnetic bead or PVP. In preferred embodiments, the recognition complex system of the present invention employs organic semiconductor chip technology wherein biological sensors are distributed across the surface of the chip so as to form an array of recognition complexes. In other embodiments, the recognition complexes of the present invention may be attached to a surface for use in a flow cell apparatus.

In certain embodiments, the biological sensors are attached to magnetic beads instead of to a chip. An array of biological sensors may be assembled, each attached to a magnetic bead. In certain embodiments, each biological sensor attached to a single magnetic bead has the same nucleic acid sequence, while in other embodiments a single magnetic bead may be attached to biological sensors of different sequences. In a preferred embodiment, the magnetic bead is attached to an organic semiconductor, preferably DAT or DALM, and the biological sensor is attached to the organic semiconductor, forming an array of recognition complexes. Although any method may be employed within the scope of the present invention to attach the organic semiconductor to the magnetic bead and the biological sensor to the organic semiconductor, in a preferred embodiment the organic semiconductor is covalently attached to the magnetic bead and the biological sensor is non-covalently attached to the organic semiconductor. In a more preferred embodiment, the attachment of biological sensor to organic semiconductor is an electrostatic interaction, preferably mediated by magnesium ion.

In certain embodiments, an array of recognition complexes attached to magnetic beads is exposed to an analyte and binding of analyte to biological sensor may be detected, for example, by photochemical changes in the biological sensor/organic semiconductor couplet upon binding to the analyte. The skilled artisan will realize that magnetic beads would be particularly useful for separating recognition complexes that bind to the analyte from recognition complexes that do not bind the analyte. In one embodiment, a magnetic flow cell, such as is described in U.S. Pat. No. 5,972,721 (incorporated herein by reference), could be used in conjunction with the recognition complex system to identify and separate analyte-binding recognition complexes from recognition complexes that do not bind the analyte.

In certain preferred embodiments, flow cytometry is used to separate recognition complexes that bind to an analyte from those that do not bind. In such embodiments, the recognition complex may be attached to a glass or other bead, or the analyte may comprise a population of cells, spores or other large particles for analytical or preparative procedures. Biological sensors that bind to the target analyte, or analytes that bind to a specific biological sensor, may be sorted, for example, by screening particles for organic semiconductor-associated fluorescence in a flow cytometer.

In another embodiment, biological sensors that bind to the analyte with high affinity can be reproduced (synthesized or amplified) for use as a neutralizing agent to inactivate or destroy the analyte. A high affinity biological sensor may be attached to a variety of agents that could be used to neutralize the analyte, such as toxic proteins, enzymes capable of activating protoxins, or other molecules or reactive moieties including radioisotopes and other organic or inorganic compounds. In certain embodiments, the high affinity biological sensor can be attached to an organic semiconductor, such as DAT or DALM. The organic semiconductor/biological sensor couplet, after binding to the analyte, may be activated by a variety of techniques, including exposure to sunlight, heat, or irradiation of various types, including laser, microwave, radiofrequency, ultraviolet and infrared. Activation of the organic semiconductor/biological sensor couplet results in absorption of energy, which may be transmitted to the analyte, inactivating or destroying it.

In other embodiments, high affinity biological sensors may be produced that can act as inhibitors, activators or binding factors of target analytes, such as a specific enzyme, receptor protein, transport protein, binding protein, cytokine, transcription factor, protein kinase, structural protein, hormone, growth factor, cell adhesion factor, angiogenic agent, nucleic acid or lipid. In this case, the ability of the high affinity biological sensor to alter the activity of the target analyte may be determined by standard techniques known in the art. For example, a biological sensor that inhibits or activates a selected target enzyme may be identified by comparing the enzyme's activity in the presence or absence of the biological sensor. A biological sensor that activates or inhibits the activity of a regulatory molecule, such as a hormone, growth factor or cytokine may be identified by bioassay, wherein a cell responsive to the hormone, growth factor or cytokine is exposed to that molecule in the presence and absence of the biological sensor.

In a preferred embodiment, the target analyte is a matrix metalloproteinase or an analogue of a matrix metalloproteinase. Production of matrix metalloproteinases is discussed in U.S. Pat. No. 6,114,159. High affinity biological sensors prepared against a matrix metalloproteinase may be assayed for their ability to inhibit metalloproteinase activity by monitoring metalloproteinase catalyzed breakdown of collagen in the presence and absence of the biological sensor, as discussed in U.S. Pat. Nos. 6,117,869, 6,118,001 and 6,124,333, each incorporated herein by reference. Biological sensors that can inhibit metalloproteinase activity may be of use to block tumor angiogenesis, or to inhibit collagen breakdown in rheumatoid arthritis or osteoarthritis.

In another preferred embodiment, the target analyte is tumor necrosis factor alpha (TNFα). Biological sensors that inhibit TNFα activity may be assayed as disclosed in U.S. Pat. Ser. No. 6,143,866, incorporated herein by reference.

In another embodiment, recognition complexes containing high affinity biological sensors may be used as biosensors to screen potential inhibitors or activators of a specific enzyme, receptor protein, transport protein, binding protein, cytokine, transcription factor, protein kinase, structural protein, hormone, growth factor, cell adhesion factor, angiogenic agent, nucleic acid or lipid for a desired biological activity. For example, a recognition complex containing a biological sensor that is specific for a catalytic product of matrix metalloproteinase (i.e., collagen breakdown product) may be prepared as discussed above. Microtiter wells containing matrix metalloproteinase, a collagen substrate, and one or more putative inhibitors may be prepared. A recognition complex that binds to a collagen breakdown product is added to each well and binding of the recognition complex to its target is analyzed by fluorescence spectroscopy or equivalent assay. The presence of an inhibitor of matrix metalloproteinase is indicated by the absence of recognition complex:analyte binding in a particular microtiter well. The skilled artisan will realize that the utility of biological sensors as biosensors is not limited to the present example, but can include any application where a biological sensor with high affinity for the product of a biological reaction may be prepared.

In certain preferred embodiments, the biological sensors of interest may be synthesized, chemically modified or selected to exhibit multiple functional properties. As non-limiting examples of multifunctional biological sensors, a biological sensor that exhibited high affinity for tumor cells could be chemically modified to attach a tumoricidal or cytotoxic agent. A biological sensor with high affinity for one target analyte could be coupled to another biological sensor with high affinity for a different analyte, to form a bridging moiety that would bind to both target analytes and hold them in close proximity. Such a multifunctional biological sensor could be designed, for example, to attach a cell surface receptor with a gene therapy vector to provide a targeting function for gene therapy. A biological sensor could be selected to exhibit a particular catalytic activity, for example by cloning and expressing the biological sensor in an appropriate host cell and screening clones for the catalytic activity of interest. Alternatively, an in vitro coupled assay could be developed to screen for catalytic activity in a biological sensor. Such a catalytic biological sensor could be attached to another biological sensor with high affinity for a desired target analyte. For example, a biological sensor with high affinity for tumor cells could be attached to a catalytic biological sensor that could convert a protoxin into an active toxin. Alternatively, a biological sensor with a desired binding specificity could be modified to attach a photoaffinity or chemical cross-linking moiety.

In certain embodiments, the high affinity biological sensor could be incorporated into an apparatus capable of being carried into the field. For example, the high affinity biological sensor could be incorporated into a patch or card to be worn by an individual. Exposure of the individual to the specific analyte for which the biological sensor exhibits high affinity could be indicated by a color change of the patch, or by a change in the photochemical properties of a biological sensor/organic semiconductor couplet. Alternatively, the high affinity biological sensor could be incorporated into an apparatus to be carried by a vehicle that could be used to cover a wide area to detect and identify unknown chemical or biological agents.

The skilled artisan will realize that the scope of the present invention is not limited to applications in chemical or biological warfare, but rather includes a broad variety of potential applications in industry and medicine, where early detection and identification of exposure to chemical or biological agents is desired. Non-limiting examples of such applications include to detect explosives or illegal drugs in an airport detection system, to detect air-borne pathogens in an air conditioner monitoring system, to detect water-borne pathogens, carcinogens, teratogens or toxins in a water quality monitoring system, to detect pathogens in a hospital operating room monitoring system, to screen for pathogens in samples of human tissues or fluids, to detect allergens, pathogens or contaminants in a food production monitoring system, to detect genetically modified organisms, or to perform high through-put screening for pharmaceutical compounds.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a recognition complex system in accordance an exemplary embodiment of the present invention.

FIG. 2 illustrates another exemplary embodiment of a recognition complex system, using recognition complexes attached to magnetic beads. The flow chart illustrates the operational relationships between the components of a preferred embodiment of a recognition complex system.

FIG. 3 illustrates a process for separation of recognition complexes, comprising magnetic beads, that bind analyte from those that do not, as well as an iterative process for producing biological sensors that bind to an analyte with high affinity.

FIGS. 4A-4H shows a comparison of spatial fluorescence spectra for two different types of recognition complex systems (ligated array—FIGS. 4A-4D—versus random 60 mers—FIGS. 4E-4H) before and after addition of various analytes. The DNA arrays were electrophoresed in 10% polyacrylamide gels and fluorescence scanning was performed using an excitation of 260 nm and emission wavelength of 420 nm. The analytes used were: Whole Cholera Toxin (FIGS. 4A and 4E); SEB=Staphylococcal Enterotoxin B (FIGS. 4B and 4F); BACA gene probes=Bacillus anthracis capsular antigen gene probes (FIGS. 4C and 4D); G10 mer (FIG. 4G) and N6-20=a DNA ladder standard composed of small DNA fragments from 6 to 20 bp (FIG. 4H).

FIG. 5 shows the reproducibility of the fluorescence emission profile of a ligated biological sensor array in a polyacrylamide gel after addition of whole cholera toxin, in the presence and absence of DALM.

FIG. 6 shows the destruction of an anthrax spore using DALM and a high power microwave pulse.

### DESCRIPTION OF ILLUSTRATIVE EMBODIMENTS

Definitions

As used herein, “a” or “an” may mean one or more than one of an item.

“Biological sensor” means a molecule having a desirable action on a target. A desirable action includes, but is not limited to, binding of the target, catalytically changing the target, reacting with the target in a way that modifies or alters the target or the functional activity of the target, covalently attaching to the target, facilitating the reaction between the target and another molecule, and neutralizing the target. In a preferred embodiment, the action is specific binding affinity for a target molecule, such target molecule being a three dimensional chemical structure. In preferred embodiments, the biological sensors are DNA, although other molecules such as RNA or modified nucleic acids are contemplated.

“Nucleic acid” means either DNA, RNA, single-stranded, double-stranded or triple stranded and any chemical modifications thereof. Virtually any modification of the nucleic acid is contemplated by this invention. Non-limiting examples of nucleic acid modifications are discussed in further detail below. “Nucleic acid” encompasses, but is not limited to, oligonucleotides and polynucleotides. “Oligonucleotide” refers to at least one molecule of between about 3 and about 100 nucleotides in length. “Polynucleotide” refers to at least one molecule of greater than about 100 nucleotides in length. These terms generally refer to at least one single-stranded molecule, but in certain embodiments also encompass at least one additional strand that is partially, substantially or fully complementary in sequence. Thus, a nucleic acid may encompass at least one double-stranded molecule or at least one triple-stranded molecule that comprises one or more complementary strand(s) or “complement(s).” As used herein, a single stranded nucleic acid may be denoted by the prefix “ss”, a double stranded nucleic acid by the prefix “ds”, and a triple stranded nucleic acid by the prefix “ts.”

Within the practice of the present invention, a “nucleic acid” may be of almost any length, from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 110, 120, 130, 140, 150, 160, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10000, 15000, 20000 or even more bases in length.

The term “nucleic acid” will generally refer to at least one molecule or strand of DNA, RNA or a derivative or mimic thereof, comprising at least one nucleobase. A “nucleobase” refers to a heterocyclic base, for example, a purine or pyrimidine base naturally found in DNA (e.g. adenine “A,” guanine “G,” thymine “T” and cytosine “C”) or RNA (e.g. A, G, uracil “U” and C), as well as their derivatives and mimics. A “derivative” refers to a chemically modified or altered form of a naturally occurring molecule, while “mimic” and “analog” refer to a molecule that may or may not structurally resemble a naturally occurring molecule, but that functions similarly to the naturally occurring molecule.

As used herein, a “moiety” generally refers to a smaller chemical or molecular component of a larger chemical or molecular structure.

A “nucleoside” is an individual chemical unit comprising a nucleobase covalently attached to a nucleobase linker moiety. An example of a “nucleobase linker moiety” is a sugar comprising 5-carbon atoms (a “5-carbon sugar”), including but not limited to deoxyribose, ribose or arabinose, and derivatives or mimics of 5-carbon sugars. Examples of derivatives or mimics of 5-carbon sugars include 2′-fluoro-2′-deoxyribose or carbocyclic sugars where a carbon is substituted for the oxygen atom in the sugar ring.

A “nucleotide” refers to a nucleoside further comprising a “backbone moiety” used for the covalent attachment of one or more nucleotides to another molecule or to each other to form a nucleic acid. The “backbone moiety” in naturally occurring nucleotides typically comprises a phosphorus moiety covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety.

“Analyte,” “target” and “target analyte” mean any compound or aggregate of interest. Non-limiting examples of analytes include a protein, peptide, carbohydrate, polysaccharide, glycoprotein, lipid, hormone, receptor, antigen, allergen, antibody, substrate, metabolite, cofactor, inhibitor, drug, pharmaceutical, nutrient, toxin, cholera toxin, Shiga-like toxin, poison, explosive, pesticide, chemical warfare agent, biohazardous agent, prion, radioisotope, vitamin, heterocyclic aromatic compound, carcinogen, mutagen, narcotic, amphetamine, barbiturate, hallucinogen, waste product, contaminant or other molecule. Molecules of any size can serve as targets. “Analytes” are not limited to single molecules, but may also comprise complex aggregates of molecules, such as a virus, bacterium, spore, mold, yeast, algae, amoebae, dinoflagellate, unicellular organism, pathogen, cell or infectious agent. In certain embodiments, cells exhibiting a particular characteristic or disease state, such as a cancer cell, may be target analytes. Virtually any chemical or biological effector would be a suitable target.

Non-limiting examples of infectious agents within the meaning of “analyte” include the following.

| | |
|---|---|
| Actinobacillus spp. | Bacillus cereus |
| Actinomyces spp. | Bacteroides spp. |
| Adenovirus (types 1, 2, 3, 4, 5 et 7) | Balantidium coli |
| Adenovirus (types 40 and 41) | Bartonella bacilliformis |
| Aerococcus spp. | Blastomyces dermatitidis |
| Aeromonas hydrophila | Bluetongue virus |
| Ancylostoma duodenale | Bordetella bronchiseptica |
| Angiostrongylus cantonensis | Bordetella pertussis |
| Ascaris lumbricoides | Borrelia burgdorferi |
| Ascaris spp. | Branhamella catarrhalis |
| Aspergillus spp. | Brucella spp. |
| Bacillus anthracis | B. abortus |
| B. canis, | Dengue virus (1, 2, 3, 4) |
| B. melitensis | Diphtheroids |
| B. suis | Eastern (Western) equine encephalitis virus |
| Brugia spp. | Ebola virus |
| Burkholderia mallei | Echinococcus granulosus |
| Burkholderia pseudomallei | Echinococcus multilocularis |
| Campylobacter fetus subsp. fetus | Echovirus |
| Campylobacter jejuni | Edwardsiella tarda |
| C. coli | Entamoeba histolytica |
| C. fetus subsp. jejuni | Enterobacter spp. |
| Candida albicans | Enterovirus 70 |
| Capnocytophaga spp. | Epidermophyton floccosum, |
| Chlamydia psittaci | Microsporum spp. Trichophyton spp. |
| Chlamydia trachomatis | Epstein-Barr virus |
| Citrobacter spp. | Escherichia coli, enterohemorrhagic |
| Clonorchis sinensis | Escherichia coli, enteroinvasive |
| Clostridium botulinum | Escherichia coli, enteropathogenic |
| Clostridium difficile | Escherichia coli, enterotoxigenic |
| Clostridium perfringens | Fasciola hepatica |
| Clostridium tetani | Francisella tularensis |
| Clostridium spp. | Fusobacterium spp. |
| Coccidioides immitis | Gemella haemolysans |
| Colorado tick fever virus | Giardia lamblia |
| Corynebacterium diphtheriae | Giardia spp. |
| Coxiella burnetii | Haemophilus ducreyi |
| Coxsackievirus | Haemophilus influenzae (group b) |
| Creutzfeldt-Jakob agent, Kuru agent | Hantavirus |
| Crimean-Congo hemorrhagic fever virus | Hepatitis A virus |
| Cryptococcus neoformans | Hepatitis B virus |
| Cryptosporidium parvum | Hepatitis C virus |
| Cytomegalovirus | Hepatitis D virus |
| Hepatitis E virus | Neisseria meningitidis |
| Herpes simplex virus | Neisseria spp. |
| Herpesvirus simiae | Nocardia spp. |
| Histoplasma capsulatum | Norwalk virus |
| Human coronavirus | Omsk hemorrhagic fever virus |
| Human immunodeficiency virus | Onchocerca volvulus |
| Human papillomavirus | Opisthorchis spp. |
| Human rotavirus | Parvovirus B19 |
| Human T-lymphotrophic virus | Pasteurella spp. |
| Influenza virus | Peptococcus spp. |
| Junin virus I Machupo virus | Peptostreptococcus spp. |
| Kiebsiella spp. | Plesiomonas shigelloides |
| Kyasanur Forest disease virus | Powassan encephalitis virus |
| Lactobacillus spp. | Proteus spp. |
| Legionella pneumophila | Pseudomonas spp. |
| Leishmania spp. | Rabies virus |
| Leptospira interrogans | Respiratory syncytial virus |
| Listeria monocyto genes | Rhinovirus |
| Lymphocytic choriomeningitis virus | Rickettsia akari |
| Marburg virus | Rickettsia prowazekii, R. canada |
| Measles virus | Rickettsia rickettsii |
| Micrococcus spp. | Ross river virus/O'Nyong-Nyong virus |
| Moraxella spp. | Rubella virus |
| Mycobacterium spp. | Salmonella choleraesuis |
| Mycobacterium tuberculosis, M. bovis | Salmonella paratyphi |
| Mycoplasma hominis, M. orale, M. | Salmonella lyphi |
| salivarium, M. fermentans | Salmonella spp. |
| Mycoplasma pneumoniae | Schistosoma spp. |
| Naegleria fowleri | Scrapie agent |
| Necator americanus | Serratia spp. |
| Neisseria gonorrhoeae | Shigella spp. |
| Sindbis virus | Yersinia pestis |
| Sporothrix schenckii | |
| St. Louis encephalitis virus | |
| Murray Valley encephalitis virus | |
| Staphylococcus aureus | |
| Streptobacillus moniliformis | |
| Streptococcus agalactiae | |
| Streptococcus faecalis | |
| Streptococcus pneumoniae | |
| Streptococcus pyogenes | |
| Streptococcus salivarius | |
| Taenia saginata | |
| Taenia solium | |
| Toxocara canis, T. cati | |
| Toxoplasma gondii | |
| Treponema pallidum | |
| Trichinella spp. | |
| Trichomonas vaginalis | |
| Trichuris trichiura | |
| Trypanosoma brucei | |
| Ureaplasma urealyticum | |
| Vaccinia virus | |
| Varicella-zoster virus | |
| Venezuelan equine encephalitis | |
| Vesicular stomatitis virus | |
| Vibrio cholerae, serovar 01 | |
| Vibrio parahaemolyticus | |
| Wuchereria bancrofti | |
| Yellow fever virus | |
| Yersinia enterocolitica | |
| Yersinia pseudotuberculosis | |

“Binding” refers to an interaction or binding between a target and a biological sensor, resulting in a sufficiently stable complex so as to permit separation of biological sensor:target complexes from uncomplexed biological sensors under given binding or reaction conditions. Binding is mediated through hydrogen bonding, electrostatic interaction, hydrophobic interaction, Van der Walls forces or other molecular forces. In certain embodiments, binding may be covalent, for example where the biological sensor or analyte contains a photoreactive or chemically reactive moiety to promote covalent attachment of biological sensor and analyte. Covalent binding may be desirable, for example, where an analyte or biological sensor is labeled to facilitate purification of the analyte: biological sensor pair.

“Organic semiconductor” means a conjugated (alternating double and single bonded) organic compound in which regions of electrons and the absence of electrons (holes or positive charges) can move with varying degrees of difficulty through the aligned conjugated system. Organic semiconductors of use in the practice of the instant invention may be fluorescent, phosphorescent, luminescent, chemiluminescent, or may be otherwise characterized by their absorption, reflection or emission of electromagnetic radiation, including infrared, ultraviolet or visible light.

“Recognition complex” refers to a biological sensor that is operably coupled to an organic semiconductor. “Operably coupled” means that the biological sensor and the organic semiconductor are in close physical proximity to each other, such that binding of an analyte to the biological sensor results in a change in the properties of the organic semiconductor that is detectable as a signal. In preferred embodiments, the signal is a photochemical signal, such as a photochemical signal, a fluorescent signal, a luminescent signal, or a change of color. In one preferred embodiment, the signal is a change in the fluorescence emission profile of the organic semiconductor/biological sensor couplet. Operable coupling may be accomplished by a variety of interactions, including but not limited non-covalent or covalent binding of the organic semiconductor to the biological sensor. In another embodiment, the biological sensor may be at least partially embedded in the organic semiconductor. Virtually any type of interaction between the organic semiconductor and the biological sensor is contemplated within the scope of the present invention, so long as the binding of an analyte to the biological sensor results in a change in the properties of the organic semiconductor. In one preferred embodiment, the biological sensor is electrostatically linked to the organic semiconductor by a magnesium ion bridge. In an alternate embodiment, the biological sensor is covalently linked to the semiconductor by chemical cross-linking. A number of suitable chemical cross-linking reagents are well known in the art, such as EDC (1-ethyl-3-(2-dimethylaminopropyl)carbodiimide).

A “recognition complex system” comprises an array of recognition complexes. In preferred embodiments, the array of recognition complexes is operably coupled to a detection unit, such that changes in the photochemical properties of the organic semiconductor that result from binding of analyte to biological sensor may be detected by the detection unit. It is contemplated within the scope of the present invention that detection may be an active process or a passive process. For example, in embodiments where the array of recognition complexes is incorporated into a card or badge, the binding of analyte may be detected by a change in color of the card or badge. In other embodiments, detection occurs by an active process, such as scanning the fluorescence emission profile of an array of recognition complexes.

“Photochemical” means any light related or light induced chemistry. A “photochemical signal” specifically includes, but is not limited to, a fluorescent signal, a luminescent signal, a phosphorescent signal or a change of color.

“Magnetic bead,” “magnetic particle” and “magnetically responsive particle” are used herein to mean any particle dispersible or suspendable in aqueous media, without significant gravitational settling and separable from suspension by application of a magnetic field. The particles comprise a magnetic metal oxide core, often surrounded by an adsorptively or covalently bound sheath or coat bearing functional groups to which various molecules, such as organic semiconductor or DNA, may be covalently coupled or adsorbed.

In certain embodiments, non-magnetic beads, such as functionalized or non-functionalized glass, or functionalized or non-functionalized polystyrene, may be used as surfaces for the attachment of recognition complexes and the separation of recognition complexes bound to analyte from complexes that do not bind analyte.

Recognition Complex System

An embodiment of the instant invention relates to compositions and apparatus capable of undergoing a process that selectively amplifies biological sensors that bind to a target analyte. This recognition complex system comprises an array of recognition complexes, each recognition complex comprising a biological sensor. In various embodiments, the biological sensor may be attached to an organic semiconductor, such as DAT or DALM. In certain embodiments, the recognition complexes are arranged in a two-dimensional array, which may be attached to a glass or other flat surface. In other embodiments, the recognition complexes comprise biological sensors attached to magnetic bead or to non-magnetic beads, such as glass, polystyrene, or polyacrylamide beads, in a three-dimensional array. In a preferred embodiment, the beads are suspended in a liquid medium.

The array of recognition complexes is exposed to analyte. Binding of analyte to individual recognition complexes is detected by, for example, changes in the photochemical properties of the recognition complex upon binding to the analyte. Where the recognition complexes comprise an organic semiconductor, such as DAT or DALM, the changes in photochemical properties may be detected by a variety of techniques, described in detail below.

Embodiments Involving A Chip Type of Array

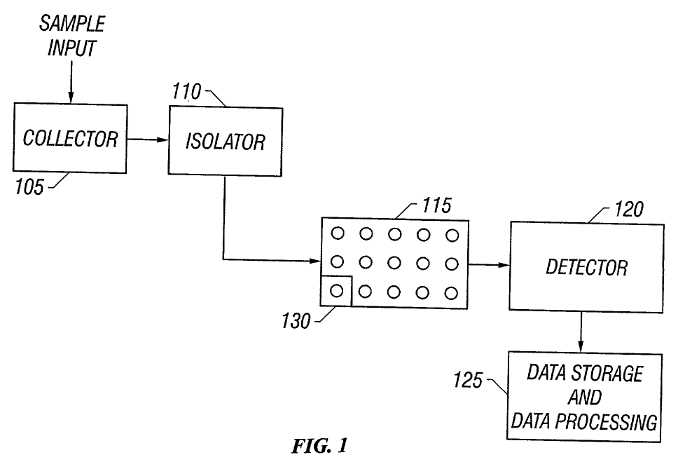

FIG. 1 illustrates a recognition complex system in accordance with an exemplary embodiment of the present invention. This embodiment of the recognition complex system includes a sample collection unit 105, an analyte isolation unit 110, an organic semiconductor chip based array of recognition complexes 115, a detection unit 120 and a data storage and processing unit 125. In general, the sample collection unit 105 is employed to actively collect or passively receive samples containing the unknown analyte to be identified. The analyte isolation unit 110 is employed to filter the sample and isolate the unknown analyte from other substances or compounds that might be present in the sample. The sample collection unit 105 and the analyte isolation unit 110 may be implemented in accordance with any number of known techniques and/or components known in the art.

The array of recognition complexes 115 comprises one or more individual recognition complexes 130. It will be understood that the array of recognition complexes 115 is shown as comprising 15 recognition complexes for illustrative purposes only. In actuality, the array 115 may contain significantly more than 15 recognition complexes. Within the scope of the invention, the array may comprise approximately 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 125, 130, 140, 150, 160, 170, 175, 180, 185, 190, 200, 225, 250, 275, 300, 325, 350, 375, 400, 425, 450, 475, 500, 550, 600, 650, 700, 750, 800, 850, 900, 950, 1000, 1500, 2000, 2500, 3000, 3500, 4000, 4500, 5000, 6000, 7000, 8000, 9000, 10000, 15000, 20000, 30000, 40000, 50000, 75000, 10000, 20000, 30000, 40000, 50000, 100000, 200000, 500000, 10⁶, 10⁷, 10⁸, 10⁹, 10¹⁰, 10¹¹, 10¹², 10¹⁴, 10¹⁶, 10¹⁸ recognition complexes or any number in between. In certain embodiments, the biological sensor component of each recognition complex differs in sequence from the biological sensor component of the other recognition complexes in the array. In other embodiments, some or all of the biological sensors may be similar or identical in sequence.

Each of the recognition complexes 130 associated with the array 115 comprises a biological sensor/organic semiconductor couplet. In preferred embodiments, the organic semiconductor is DAT. In alternative embodiments, the organic semiconductor used in diazoluminomelanin (DALM). DAT and DALM are polymers that exhibit slow fluorescent, chemiluminescent, sonochemiluminescent, thermochemiluminescent and electrochemi- luminescent properties. However, other organic semiconductors may serve as acceptable substitutes.

As shown in FIG. 1, the recognition complex system comprises an array 115 of recognition complexes, such as recognition complex 130. Each of these recognition complexes comprises a biological sensor/organic semiconductor couplet. Separating each of the recognition complexes is binding material. The biological sensor sequences present at each of the recognition complexes may be random sequences.

After collecting and isolating the unknown analyte, the analyte is applied to each recognition complex associated with the array 115. In those embodiments where the biological sensor sequences are not identical, some of the biological sensors will exhibit a high affinity for the analyte, some biological sensors will exhibit less affinity for the analyte and some biological sensors will exhibit no affinity for the analyte. The photochemical properties of the biological sensor/organic semiconductor couplet will change depending on the degree to which the biological sensors bind to the analyte. The photochemical properties associated with some recognition complexes will change significantly, while the photochemical properties associated with other recognition complexes may change very little, if at all, upon exposure to a given analyte.

The photochemical changes may be detected by the detection unit 120. In preferred embodiments, the detection unit 120 comprises a charge coupled device (CCD), such as a CCD camera, digital camera, photomultiplier tube or any other functionally equivalent detector.

The photochemical signature of the analyte may consist of a two-dimensional distribution of fluorescence resulting from long-wavelength ultraviolet light excitation. Response of the array 115 at a specific spatial location 130 may be similar for two or more different analytes, but by combining the fluorescence response of many independent measurement locations, specificity can be high. A typical consumer-type CCD-based color video camera has 768×494 discrete detectors. A miniaturized cell utilizing such a camera with a array could have about 380,000 parallel channels (single detectors). Practical considerations would group detectors for lower but less spatially noisy resolution with fewer channels. Hundreds to thousands of channels could easily be achieved. Optimization of the number of channels would minimize channels and thus computational load, while maximizing specificity and classification accuracy.

Analysis of the photochemical signature, by data processing unit 125, may involve a comparison of multiple channels of fluorescence spectral signatures. Use of CIE colorimetry methods may streamline processing by representing spectral distributions at each spatial location as CIE chromaticity coordinates (two numbers). Such methods also provide an analytical technique that is color oriented and relatively independent of intensity. Comparison of signatures by data processing unit 125 may be implemented using artificial neural networks (such as the Qnet v2000 neural net software package from Vesta Services, Inc., 1001 Green Bay Rd., Winnetka, Ill. 60093), look-up tables or various other decision methods, operating on the arrays of two-number (CIE chromaticity) coordinates that are the signatures for identified analytes. This would provide a fast comparison of unknown analytes to a database of previously recorded signatures of known analytes.

In accordance with one aspect of the present invention, unknown chemical and biological analytes may be detected and identified in a single, automated binding step, as the reaction between the analyte and the biological sensor sequences distributed across the array 115 produces a relatively unique change in the photochemical properties of the array as a whole. However, where two or more analytes share similar chemical structures, they might cause the array 115 to produce a relatively similar photochemical response.

Thus, in accordance with another aspect of the present invention, a more unique photochemical response from the array 115 can be achieved to more clearly distinguish between structurally similar analytes. To accomplish this, the biological sensors associated with those recognition complexes that bind to the analyte, as indicated by changes in photochemical properties, may extracted from the array.

In certain embodiments, individual recognition complexes 130 may be detached from the array 115 by hydrolysis, cleavage, heating or other methods of dissociation applied to the array at the location of each such recognition complex. The biological sensor sequences exhibiting affinity for analyte may be separated from the analyte by washing the biological sensor bound to analyte with deionized water, salt solutions, detergents, chaotrophic agents, solvents or other solutions that serve to separate the analyte from biological sensor. The biological sensor sequences that exhibit no affinity for the analyte can be discarded. The extracted biological sensor sequences may be amplified and applied to a clean chip to produce a new array 115. Since the new array 115 comprises only those biological sensor sequences that were identified as binding to the analyte, it should exhibit a greater degree of specificity and a higher binding affinity for the analyte.

Once a new array chip 115 is produced, analyte may be introduced to each of the array recognition complexes 130, and the photochemical changes across the array may be detected and analyzed, producing an even more unique signature that can be used for analyte identification and to distinguish the analyte from chemically or structurally similar species.

The production of chips for attachment of biological sensors is well known in the art. The chip may comprise a Langmuir-Bodgett film, functionalized glass, germanium, silicon, PTFE, polystyrene, gallium arsenide, gold, silver, membrane, nylon, PVP, or any other material known in the art that is capable of having functional groups such as amino, carboxyl, Diels-Alder reactants, thiol or hydroxyl incorporated on its surface. In certain embodiments,, these groups may be covalently attached to cross-linking agents so that binding interactions between analyte and recognition complex occur without steric hindrance from the chip surface. Typical cross-linking groups include ethylene glycol oligomer, diamines and amino acids. Any suitable technique useful for immobilizing a recognition complex on a chip is contemplated by this invention, including sialinization. In preferred embodiments, the organic semiconductor is attached to the chip surface and biological sensors are then attached, covalently or non-covalently, to the organic semiconductor.

The array-based chip design 115 may be distinguished from conventional biochips (e.g., U. S. Pat. Nos. 5,861,242 and 5,578,832) by a number of characteristics, including the use of an organic semiconductor, such as DAT or DALM. Additionally, conventional biochips typically are constructed by attaching or synthesizing biological sensors having affinities for known analytes on specific identified locations on the chip. The presence of a target analyte in a sample is detected by binding to the specific chip locus containing a biological sensor with known affinity for that analyte. In contrast, in certain embodiments of the present invention the affinities of the biological sensor/organic semiconductor couplets for various analytes are unknown at the time they are initially attached to the chip. Target analytes are identified by their pattern of binding to the entire chip, not by their binding to a specific locus on the chip. This system provides greater efficiency and flexibility, in that it is not necessary to prepare biological sensors of known specificity before construction of the chip. Further, previously unknown analytes may be characterized by their pattern of interaction with the chip, without having to clone and sequence their RNA or DNA or prepare high-affinity biological sensors in advance of chip production.

Embodiments Involving Magnetic Beads

In an alternative embodiment, the biological sensor sequences may be attached to magnetic beads instead of to a glass or other flat surface. In this case, each recognition complex would comprise a magnetic bead attached to one or more biological sensors. In a preferred embodiment, each biological sensor molecule attached to the same magnetic bead will have the same sequence. In other embodiments, the biological sensor molecules attached to a single bead may have different sequences. In certain preferred embodiments, the biological sensors will also be attached to an organic semiconductor. Attachment of biological sensors to organic semiconductors would facilitate the detection and quantitation of analyte binding to the biological sensors, as described above.

The skilled artisan will realize that use of magnetic bead technology would facilitate certain applications of the invention, such as the iterative process for producing biological sensors of higher specificity and greater binding affinity for the analyte. With magnetic bead technology, the individual recognition complexes are more easily manipulated and separated according to their characteristics. For example, recognition complexes that bind to the analyte may be separated from recognition complexes that do not bind to the analyte by using a magnetic flow cell or filter block, as disclosed in U.S. Pat. No. 5,972,721, incorporated herein by reference in its entirety.

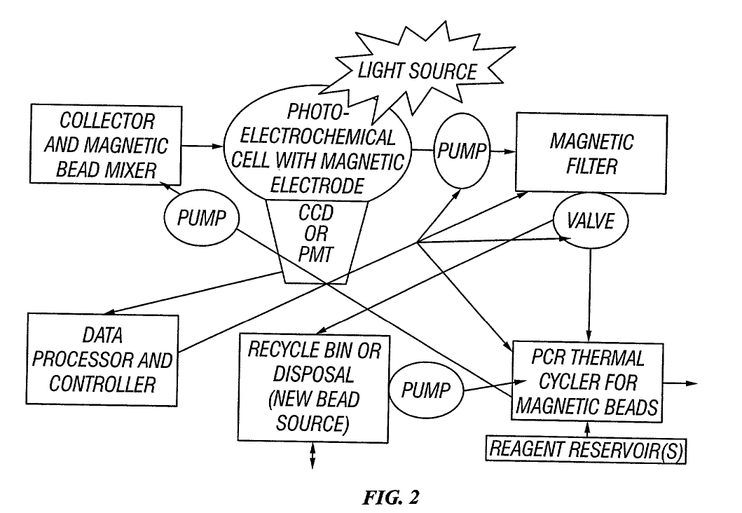

A diagram for use of magnetic beads in a recognition complex system is shown in FIG. 2. Biological sensors of random or non-random sequence may be synthesized or amplified and attached to magnetic beads. The individual recognition complexes, each corresponding to a magnetic bead attached to one or more biological sensors, together comprise an array, similar to that described above for FIG. 1. The array is added to the magnetic bead mixer (FIG. 2) and analyte is added and allowed to bind to the biological sensors. The mixture is then transferred to a photo-photochemical cell with a magnetic electrode, where the mixture may be exposed to ultraviolet or other irradiation. A CCD, photomultiplier tube, digital camera or other detection device may be used to obtain absorption or emission spectra. As described above, binding of analyte will result in characteristic changes in the photochemical properties of individual recognition complexes. These changes in photochemical properties will be detected and analyzed to produce an analyte signature, as described above. Although the suspension of recognition complexes in the bead mixer is random, the use of a magnetic electrode in the photo-photochemical cell will provide a spatial distribution of recognition complexes, analogous to the two-dimensional array 115 described above. Beads will deposit and separate on the surface of the magnetic electrode according to their accumulated mass (from binding analyte). This spatial distribution, along with the detected photochemical changes, may be analyzed to produce a unique signature that can be used to identify the analyte.

Certain components that may be incorporated into a recognition complex system as shown in FIG. 2 include pumps and valves to facilitate fluid transfer between different components of the recognition complex system. It is anticipated that virtually any pump or valve capable of producing a controlled fluid transfer between one component and another component of the recognition complex system illustrated in FIG. 2 could be used.

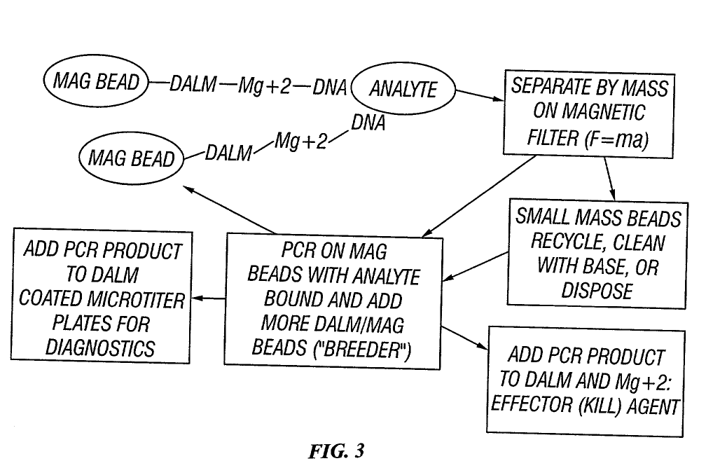

Processes for the coupling of molecules to magnetic beads or a magnetite substrate are well known in the art, i.e. U.S. Pat. Nos. 4,695,393, 3,970,518, 4,230,685, and 4,677,055 herein expressly incorporated by reference. Alternatively, an organic semiconductor may be attached directly to the magnetic bead. Biological sensors, such as DNA, may be attached to the organic semiconductor by electrostatic interaction with magnesium ion (FIG. 3). This would facilitate detachment of DNA from the organic semiconductor/magnetic bead, since DNA would be released by addition of a chelating agent such as EDTA (ethylene diamine tetraacetic acid). Alternatively, the biological sensor may be covalently attached, for example by chemical cross-linking to the organic semiconductor through the use of any appropriate cross-linking agent known in the art, such as EDC.

It is envisioned that particles employed in the instant invention may come in a variety of sizes. While large magnetic particles (mean diameter in solution greater than 10 μm) can respond to weak magnetic fields and magnetic field gradients, they tend to settle rapidly, limiting their usefulness for reactions requiring homogeneous conditions. Large particles also have a more limited surface area per weight than smaller particles, so that less material can be coupled to them. In preferred embodiments, the magnetic beads are less than 10 μm in diameter.

Various silane couplings applicable to magnetic beads are discussed in U.S. Pat. No. 3,652,761, incorporated herein by reference. Procedures for silanization known in the art generally differ from each other in the media chosen for the polymerization of silane and its deposition on reactive surfaces., Organic solvents such as toluene (Weetall, (1976)), methanol, (U.S. Pat. No. 3,933,997) and chloroform (U.S. Pat. No. 3,652,761) have been used. Silane deposition from aqueous alcohol and aqueous solutions with acid have also been used.

Ferromagnetic materials in general become permanently magnetized in response to magnetic fields. Materials termed “superparamagnetic” experience a force in a magnetic field gradient, but do not become permanently magnetized. Crystals of magnetic iron oxides may be either ferromagnetic or superparamagnetic, depending on the size of the crystals. Superparamagnetic oxides of iron generally result when the crystal is less than about 300 angstroms (Å) in diameter; larger crystals generally have a ferromagnetic character.

Dispersible magnetic iron oxide particles reportedly having 300 Å diameters and surface amine groups were prepared by base precipitation of ferrous chloride and ferric chloride (Fe²⁺/Fe³⁺=1) in the presence of polyethylene imine, according to U.S. Pat. No. 4,267,234. These particles were exposed to a magnetic field three times during preparation and were described as redispersible. The magnetic particles were mixed with a glutaraldehyde suspension polymerization system to form magnetic polyglutaraldehyde microspheres with reported diameters of 0.1 μm. Polyglutaraldehyde microspheres have conjugated aldehyde groups on the surface which can form bonds to amino containing molecules such as proteins.

While a variety of particle sizes are envisioned to be applicable in the disclosed method, in a preferred embodiment, particles are between about 0.1 and about 1.5 μm diameter. Particles with mean diameters in this range can be produced with a surface area as high as about 100 to 150 m²/gm, which provides a high capacity for bioaffinity adsorbent coupling. Magnetic particles of this size range overcome the rapid settling problems of larger particles, but obviate the need for large magnets to generate the magnetic fields and magnetic field gradients required to separate smaller particles. Magnets used to effect separations of the magnetic particles of this invention need only generate magnetic fields between about 100 and about 1000 Oersteds. Such fields can be obtained with permanent magnets that are preferably smaller than the container which holds the dispersion of magnetic particles and thus, may be suitable for benchtop use. Although ferromagnetic particles may be useful in certain applications of the invention, particles with superparamagnetic behavior are usually preferred since superparamagnetic particles do not exhibit the magnetic aggregation associated with ferromagnetic particles and permit redispersion and reuse.

The method for preparing the magnetic particles may comprise precipitating metal salts in base to form fine magnetic metal oxide crystals, redispersing and washing the crystals in water and in an electrolyte. Magnetic separations may be used to collect the crystals between washes if the crystals are superparamagnetic. The crystals may then be coated with a material capable of adsorptively or covalently bonding to the metal oxide and bearing functional groups for coupling with biological sensors or organic semiconductors.

Embodiments Involving Non-Magnetic Beads, Cells or Particles and Flow Cytometry

In another embodiment, the recognition complexes or analyte of interest may be non-covalently or covalently attached to non-magnetic beads, such as glass, polyacrylamide, polystyrene or latex. Receptor complexes may be attached to such beads by the same techniques discussed above for magnetic beads. After exposure of analyte to receptor complexes, those complexes bound to analyte may be separated from unbound complexes by flow cytometry. Non-limiting examples of flow cytometry methods are disclosed in Betz et al. (1984), Wilson et al. (1988), Scillian et al. (1989), Frengen et al. (1994), Griffith et al. (1996), Stuart et al. (1998) and U.S. Pat. Nos. 5,853,984 and 5,948,627, each incorporated herein by reference in its entirety. U.S. Pat. Nos. 4,727,020, 4,704,891 and 4,599,307, incorporated herein by reference, describe the arrangement of the components comprising a flow cytometer and the general principles of its use.

In the flow cytometer, beads, cells or other particles are passed substantially one at a time through a detector, where each particle is exposed to an energy source. The energy source generally provides excitatory light of a single wavelength. The detector comprises a light collection unit, such as photomultiplier tubes or a charge coupled device, which may be attached to a data analyzer such as a computer. The beads, cells or particles can be characterized by their response to excitatory light, for example by detecting and/or quantifying the amount of fluorescent light emitted in response to the excitatory light. Changes in size due to binding of analyte to biological sensor can also be incorporated into sorting strategies. Beads or cells exhibiting a particular characteristic can be sorted using an attached cell sorter, such as the FACS Vantage ™ cell sorter sold by Becton Dickinson Immunocytometry Systems (San Jose, Calif.).

This system is well suited to use with an organic semiconductor that has well defined fluorescent and luminescent properties. Using a flow cytometer, it is possible to separate beads, cells or particles that are associated with recognition complexes bound to analytes, from unbound complexes, by detecting the presence of and characterizing the photochemical properties of the organic semiconductor. Because those properties change upon binding of recognition complex to analyte, it is possible to separate bead-attached recognition complexes that bind to analyte from complexes that do not bind analyte. This process is even simpler when the analyte is incorporated into a cell or cell fragment, or attached to a bead. In this case, only analytes bound to recognition complexes should show a fluorescent or other spectroscopic signature associated with the organic semiconductor. In an alternative embodiment, the analyte or biological sensor may be labeled with a different fluorescent or other spectroscopic tag moiety. Many examples of fluorescent or other tag moieties are known in the art.

Flow cytometry may be used to purify or partially purify analytes that bind to a particular biological sensor, or to purify or partially purify biological sensors that bind to a particular analyte. Other manipulations may include sorting for differences in fluorescence and/or size that represent differences in binding affinity or avidity of analyte for biological sensor or the number of biological sensors bound to each analyte or of analyte bound to each biological sensor.

Biological Sensors

Biological sensors within the scope of the present invention may be made by any technique known to one of ordinary skill in the art. Non-limiting examples of biological sensors include synthetic oligonucleotides made by in vitro chemical synthesis using phosphotriester, phosphite or phosphoramidite chemistry and solid phase techniques (EP 266,032, incorporated herein by reference) or via deoxynucleoside H-phosphonate intermediates (Froehler et al., 1986, and U.S. Pat. Ser. No. 5,705,629, each incorporated herein by reference). Examples of enzymatically produced biological sensors include those produced by amplification reactions such as PCR™ (e.g., U.S. Pat. No. 4,683,202 and U.S. Pat. No. 4,683,195, each incorporated herein by reference), or the synthesis of oligonucleotides described in U.S. Pat. No. 5,645,897, incorporated herein by reference. Examples of a biologically produced biological sensor include recombinant nucleic acid production in living cells, such as recombinant DNA vector production in bacteria (e.g., Sambrook et al. 1989).

Nucleobase, nucleoside and nucleotide mimics or derivatives are well known in the art, and have been described in exemplary references such as, for example, Scheit, Nucleotide Analogs (John Wiley, New York, 1980). Purine and pyrimidine nucleobases encompass naturally occurring purines and pyrimidines and derivatives and mimics thereof. These include, but are not limited to, purines and pyrimidines substituted with one or more alkyl, carboxyalkyl, amino, hydroxyl, halogen (i.e. fluoro, chloro, bromo, or iodo), thiol, or alkylthiol groups. The alkyl substituents may comprise from about 1, 2, 3, 4, or 5, to about 6 carbon atoms.

Examples of purines and pyrimidines include deazapurines, 2,6-diaminopurine, 5-fluorouracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcytosine, 5-bromouracil, 5-ethyluracil, 5-iodouracil, 5-chlorouracil, 5-propyluracil, thiouracil, 2-methyladenine, methylthioadenine, N,N-dimethyladenine, azaadenines, 8-bromoadenine, 8-hydroxyadenine, 6-hydroxyaminopurine, 6-thiopurine, 4-(6-aminohexyl/cytosine), and the like. A list of exemplary purine and pyrimidine derivatives and mimics is provided in Table 1.

TABLE 1
Purine and Purimidine Derivatives or Mimics

| Abbr. | Modified base description | Abbr. | Modified base description |
|---|---|---|---|
| ac4c | 4-acetylcytidine | mam5s2u | 5-methoxyaminomethyl-2- |
| | | | thiouridine |
| chm5u | 5-(carboxyhydroxylmethyl)uridine | man q | Beta, D-mannosylqueosine |
| Cm | 2′-O-methylcytidine | mcm5s2u | 5-methoxycarbonylmethyl-2- |
| | | | thiouridine |
| cmnm5s2u | 5-carboxymethylaminomethyl-2- | mcm5u | 5-methoxycarbonylmethyluridine |
| | thioridine | | |
| cmnm5u | 5-carboxymethylaminomethyluridine | mo5u | 5-methoxyuridine |
| D | Dihydrouridine | ms2i6a | 2-methylthio-N6- |
| | | | isopentenyladenosine |
| Fm | 2′-O-methylpseudouridine | ms2t6a | N-((9-beta-D-ribofuranosyl-2- |
| | | | methylthiopurine-6- |
| | | | yl)carbamoyl)threonine |
| gal q | beta, D-galactosylqueosine | mt6a | N-((9-beta-D-ribofuranosylpurine- |
| | | | 6-yl)N-methyl-carbamoyl)threonine |
| Gm | 2′-O-methylguanosine | mv | Uridine-5-oxyacetic acid |
| | | | methylester |
| I | Inosine | o5u | Uridine-5-oxyacetic acid (v) |
| i6a | N6-isopentenyladenosine | osyw | Wybutoxosine |
| m1a | 1-methyladenosine | p | Pseudouridine |
| m1f | 1-methylpseudouridine | q | Queosine |
| m1g | 1 -methylguanosine | s2c | 2-thiocytidine |
| m1I | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | t | 5-methyluridine |
| m3c | 3-methylcytidine | t6a | N-((9-beta-D-ribofuranosylpurine- |
| | | | 6-yl)carbamoyl)threonine |
| m5c | 5-methylcytidine | tm | 2′-O-methyl-5-methyluridine |
| m6a | N6-methyladenosine | um | 2′-O-methyluridine |
| m7g | 7-methylguanosine | yw | Wybutosine |
| mam5u | 5-methylaminomethyluridine | x | 3-(3-amino-3- |
| | | | carboxypropyl)uridine, (acp3)u |

An example of a biological sensor comprising nucleoside or nucleotide derivatives and mimics is a “polyether nucleic acid”, described in U.S. Pat. Ser. No. 5,908,845, incorporated herein by reference, wherein one or more nucleobases are linked to chiral carbon atoms in a polyether backbone. Another example of a biological sensor is a “peptide nucleic acid”, also known as a “PNA”, “peptide-based nucleic acid mimics” or “PENAMs”, described in U.S. Pat. Ser. Nos. 5,786,461, 5,891,625, 5,773,571, 5,766,855, 5,736,336, 5,719,262, 5,714,331, 5,539,082, and WO 92/20702, each of which is incorporated herein by reference. A peptide nucleic acid generally comprises at least one nucleobase and at least one nucleobase linker moiety that is not a 5-carbon sugar and/or at least one backbone moiety that is not a phosphate group. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

Peptide nucleic acids generally have enhanced sequence specificity, binding properties, and resistance to enzymatic degradation in comparison to molecules such as DNA and RNA (Egholm et al., Nature 1993, 365, 566; PCT/EP/01219). In addition, U.S. Pat. Nos. 5,766,855, 5,719,262, 5,714,331 and 5,736,336 describe PNAs comprising nucleobases and alkylamine side chains with further improvements in sequence specificity, solubility and binding affinity. These properties promote double or triple helix formation between a target and the PNA.

The skilled artisan will realize that the present invention is not limited to the examples disclosed herein, but may include nucleobases, nucleotides and nucleic acids produced by any other means known in the art.

Amplification

In certain embodiments, the biological sensors may be amplified to provide a source of high affinity biological sensors for neutralizing analytes. Within the scope of the present invention, amplification may be accomplished by any means known in the art. Exemplary embodiments are described below.

Primers

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

Amplification Methods

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference.

Briefly, in PCR, two primer sequences are prepared which are complementary to regions on opposite complementary strands of, for example, a biological sensor. An excess of deoxynucleoside triphosphates are added to a reaction mixture along with a DNA polymerase, e.g., Taq polymerase. Examples of polymerases that may be used for purposes of nucleic acid amplification are provided in Table 2 below. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the biological sensor to form reaction products, excess primers will bind to the biological sensor and to the reaction products and the process is repeated.

The skilled artisan will realize that the methods of amplification of nucleic acid biological sensors are not limited to those listed herein, but may include any method known in the art.

Labels

For certain embodiments, it may be desirable to incorporate a label into biological sensors, probes or primers. A number of different labels may be used, such as fluorophores, chromophores, radioisotopes, enzymatic tags, antibodies, chemiluminescent, electroluminescent, affinity labels, etc. One of skill in the art will recognize that these and other label moieties not mentioned herein can be used in the practice of the present invention.

Examples of affinity labels include an antibody, an antibody fragment, a receptor protein, a hormone, biotin, DNP, and any polypeptide/protein molecule that binds to an affinity label.

Examples of enzymatic tags include urease, alkaline phosphatase or peroxidase. Colorimetric indicator substrates can be employed with such enzymes to provide a detection means visible to the human eye or spectrophotometrically.

The following fluorophores are contemplated to be useful in practicing the present invention. Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

Imaging Agents and Radioisotopes

In certain embodiments, the claimed biological sensors of the present invention may be attached to imaging agents of use for imaging, treatment and diagnosis of various diseased organs or tissues. Many appropriate imaging agents are known in the art, as are methods for their attachment to nucleic acids. Certain attachment methods involve the use of a metal chelate complex employing, for example, an organic chelating agent such a DTPA attached to the nucleic acid.

Non-limiting examples of paramagnetic ions of potential use as imaging agents include chromium (E), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²EU, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Methods of Immobilization

In various embodiments, the biological sensors of the present invention may be attached to a solid surface (“immobilized”). In a preferred embodiment, immobilization may occur by attachment of an organic semiconductor to a solid surface, such as a magnetic, glass or plastic bead, a plastic microtiter plate or a glass slide. Biological sensors may be attached to the organic semiconductor by electrostatic interaction with magnesium ion (FIG. 3). This system is advantageous in that the attachment of biological sensor to organic semiconductor may be readily reversed by addition of a magnesium chelator, such as EDTA.

Immobilization of biological sensors may alternatively be achieved by a variety of methods involving either non-covalent or covalent interactions between the immobilized biological sensor, comprising an anchorable moiety, and an anchor. In an exemplary embodiment, immobilization may be achieved by coating a solid surface with streptavidin or avidin and the subsequent attachment of a biotinylated polynucleotide (Holmstrom, 1993). Immobilization may also occur by coating a polystyrene or glass solid surface with poly-L-Lys or poly L-Lys, Phe, followed by covalent attachment of either amino- or sulfhydryl-modified polynucleotides, using bifunctional crosslinking reagents (Running, 1990; Newton, 1993).

Immobilization may take place by direct covalent attachment of short, 5′-phosphorylated primers to chemically modified polystyrene plates (“Covalink” plates, Nunc) Rasmussen, (1991). The covalent bond between the modified oligonucleotide and the solid phase surface is formed by condensation with a water-soluble carbodiimide. This method facilitates a predominantly 5′-attachment of the oligonucleotides via their 5′-phosphates.

Nikiforov et al. (U.S. Pat. No. 5,610,287 incorporated herein by reference) describes a method of non-covalently immobilizing biological sensor molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the biological sensor and the cationic detergent or salt. The support containing the immobilized biological sensor may be washed with an aqueous solution containing a non-ionic detergent without removing the attached molecules.

Another commercially available method for immobilization is the “Reacti-Bind™ DNA Coating Solutions” (see “Instructions—Reacti-Bind™ DNA Coating Solution” 1/1997). This product comprises a solution that is mixed with DNA and applied to surfaces such as polystyrene or polypropylene. After overnight incubation, the solution is removed, the surface washed with buffer and dried, after which it is ready for hybridization. It is envisioned that similar products, i.e. Costar “DNA-BIND™” or Immobilon-AV Affinity Membrane (IAV, Millipore, Bedford, Mass.) may be used in the practice of the instant invention.

Cross-linkers

Bifunctional cross-linking reagents may be of use in various embodiments of the claimed invention, such as attaching an organic semiconductor to a biological sensor, attaching an organic semiconductor to a substrate, attaching various functional groups to a biological sensor, or attaching a biological sensor or an analyte to a bead or particle. Homobifunctional reagents that carry two identical functional groups are highly efficient in inducing cross-linking. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, guanidino, indole, or carboxyl specific groups. Of these, reagents directed to free amino groups have become especially popular because of their commercial availability, ease of synthesis and the mild reaction conditions under which they can be applied. Exemplary methods for cross-linking molecules, such as organic semiconductors, biological sensors or analytes, are described in U.S. Pat. No. 5,603,872 and U.S. Pat. No. 5,401,511. Various biological sensors can be covalently bound to surfaces through the cross-linking of amine residues. Amine residues may be introduced onto a surface through the use of aminosilane, as discussed above. Coating with aminosilane provides an active functional residue, a primary amine, on the surface for cross-linking purposes. Biological sensors are bound covalently to discrete sites on the surfaces. The surfaces may also have sites for non-covalent association. To form covalent conjugates of biological sensors and surfaces, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably l-ethyl-3-(3-dimethylaminopropyl) carbodiimide (EDC). Through the complex chemistry of cross-linking, linkage of the amine residues of the silane-coated surface and free organic semiconductor, biological sensor or analyte may be accomplished.

Separation and Quantitation Methods

It may be desirable to separate biological sensors of different lengths for the purpose of quantitation, analysis or purification.

Gel Electrophoresis

In one embodiment, amplification products are separated by agarose, agarose-acrylamide or polyacrylamide gel electrophoresis using standard methods (Sambrook et al., 1989).

Separation by electrophoresis is based upon the differential migration through a gel according to the size and ionic charge of the molecules in an electrical field. High resolution techniques normally use a gel support for the fluid phase. Examples of gels used are starch, acrylamide, agarose or mixtures of acrylamide and agarose. Separated nucleic acids may be visualized by staining, for example with ethidium bromide. The gel may be a single concentration or gradient in which pore size decreases with migration distance. In gel electrophoresis of polynucleotides, mobility depends primarily on molecular size. In pulse field electrophoresis, two fields are applied alternately at right angles to each other to minimize diffusion mediated spread of large linear polymers.

Agarose gel electrophoresis facilitates the separation of DNA or RNA based upon size in a matrix composed of a highly purified form of agar. Nucleic acids tend to become oriented in an end on position in the presence of an electric field. Migration through the gel matrices occurs at a rate inversely proportional to the log₁₀ of the number of base pairs (Sambrook et al., 1989).

Chromatographic Techniques

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982). In yet another alternative, cDNA products labeled with biotin or antigen can be captured with beads bearing avidin or antibody, respectively.

Microfluidic Techniques

Microfluidic techniques include separation on a platform such as microcapillaries, designed by ACLARA BioSciences Inc., or the LabChip™ liquid integrated circuits made by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414 reports an integrated micro-PCR™ apparatus for collection and amplification of nucleic acids from a specimen. U.S. Pat. No. 5,856,174 describes an apparatus which combines the various processing and analytical operations involved in nucleic acid analysis and is incorporated herein by reference.

Capillary Electrophoresis

In some embodiments, it may be desirable to provide an additional, or alternative means for analyzing biological sensors. In these embodiment, microcapillary arrays are contemplated to be used for the analysis.

Microcapillary array electrophoresis generally involves the use of a thin capillary or channel that may or may not be filled with a particular separation medium. Electrophoresis of a sample through the capillary provides a size based separation profile for the sample. The use of microcapillary electrophoresis in size separation of nucleic acids has been reported in, e.g., Woolley and Mathies, 1994. Microcapillary array electrophoresis generally provides a rapid method for size-based sequencing, PCR™ product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Microfabrication of microfluidic devices including microcapillary electrophoretic devices has been discussed in detail in, e.g., Jacobsen et al., 1994; Effenhauser et al., 1994; Harrison et al., 1993; Effenhauser et al., 1993; Manz et al., 1992; and U.S. Pat. No. 5,904,824, incorporated herein by reference. Typically, these methods comprise photolithographic etching of micron scale channels on silica, silicon or other crystalline substrates or chips, and can be readily adapted for use in the present invention. In some embodiments, the capillary arrays may be fabricated from the same polymeric materials described for the fabrication of the body of the device, using injection molding techniques.

Tsuda et al., 1990, describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature biological sensors in the sample.

DAT

In preferred embodiments, the organic semiconductor of use in the disclosed compositions, methods and apparatus is DAT. Generally, DAT may be produced by reacting 3-amino-L-tyrosine (3AT), with an alkali metal nitrite, such as NaNO₂. In preferred embodiments, the 3AT is dissolved first in an aqueous or similar medium before reaction with NaNO₂. Surprisingly, the product of this reaction exhibits spectroscopic properties similar to DALM (U.S. Pat. No. 6,303,316). DALM is synthesized using luminol, a known luminescent compound. It was unexpected that DAT synthesized without incorporation of any luminol would show luminescent characteristics similar to DALM.

Since diazotization reactions are, in general, exothermic, in some embodiments the reaction may be carried out under isothermal conditions or at a reduced temperature, such as, for example, at ice bath temperatures. The reaction may be carried out with refluxing for 1 hour, 2 hours, 4 hours, 6 hours or preferably 8 hours, although longer reaction periods of 10, 12, 14, 18, 20 or even 24 hours are contemplated.

The DAT may be precipitated from aqueous solution by addition of a solvent in which DAT is not soluble, such as acetone. After centrifuging the precipitate and discarding the supernatant, the solid material may be dried under vacuum.

In general, the quantities of the 3AT and alkali metal nitrite reactants used are equimolar. It is, however, within the scope of the invention to vary the quantities of the reactants. The molar ratio of 3AT:metal nitrite may be varied over the range of about 0.6:1 to 3:1.

In alternative embodiments, DAT may be partially or fully oxidized prior to use, resulting in the production of oxidized-DAT (O-DAT). Reduced DAT is dissolved in 5 ml of distilled water with 0.2 gm of sodium bicarbonate added. Five milliliters of 30% hydrogen peroxide is added and the mixture is refluxed until the color of the solution changes from brown to yellow. The mixture is cooled, dialyzed against distilled water and lyophilized. The lyophilized powder contains O-DAT.

DALM

In alternative embodiments, DALM is used to attach biological sensors to a surface and/or to promote photochemical detection of binding of analyte to biological sensor. Production and use of diazoluminomelanin (DALM) has previously been described in U.S. Pat. Nos. 5,856,108 and 5,003,050, incorporated herein by reference. DALM is prepared by reacting 3AT (3-amino-L-tyrosine) with an alkali metal nitrite, such as sodium nitrite, and thereafter reacting the resulting diazotized product with luminol. At some point in the reaction, the alaninyl portion of the 3AT rearranges to provide the hydroxyindole portion of the final product. It is believed that such rearrangement occurs following coupling of the luminol to the diazotized 3AT.

The reaction between 3AT and the alkali metal nitrite is carried out in aqueous medium. Since diazotization reactions are, in general, exothermic, it may be desirable to carry out this reaction under isothermal conditions or at a reduced temperature, such as, for example, at ice bath temperatures. The reaction time for the diazotization can range from about 1 to 20 minutes, preferably about 5 to 10 minutes.

Because of the relative insolubility of luminol in aqueous medium, the luminol is dissolved in an aprotic solvent, such as dimethylsulfoxide (DMSO), then added, with stirring, to the aqueous solution of diazotized 3AT. This reaction is carried out, at reduced temperature, for about 20 to 200 minutes. The solvent is then removed by evaporation at low pressure, with moderate heating, e.g., about 30° to 37° C.

The reaction mixture is acidic, having a pH of about 3.5. The coupling of the luminol and the diazotized 3AT can be facilitated by adjusting the pH of the reaction mixture to about 5.0 to 6.0.

The product DALM may be precipitated from the reaction mixture by combining the reaction mixture with an excess of a material that is not a solvent for the DALM, e.g., acetone. After centrifuging the precipitate and discarding the supernatant, the solid material may be dried under vacuum.

In general, the quantities of the 3AT, alkali metal nitrite and luminol reactants are equimolar. It is, however, within the scope of the invention to vary the quantities of the reactants. The molar ratio of 3AT:luminol may be varied over the range of about 0.6:1 to 3:1.

DALM is water soluble, having an apparent pKa for solubility about pH 5.0. DALM does not require a catalyst for chemiluminescence. The duration of the reaction is in excess of 52 hours. In contrast, luminol requires a catalyst. With micro peroxidase as the catalyst, luminol has shown peak luminescence at 1 sec and half-lives of light emission of 0.5 and 4.5 sec at pH 8.6 and 12.6, respectively. The chemiluminescence yield of DALM is better at pH 7.4 than at pH 9.5, although it still provides a strong signal at strongly basic pHs. DALM also produces chemiluminescence at pH 6.5 which is about the same intensity as that produced at pH 9.5.

Nucleic Acid Biological Sensors

In certain preferred embodiments, the biological sensors to be used in the practice of the invention are nucleic acids. Nucleic acids may be prepared by any known method, including synthetic, recombinant, and purification methods, and may be used alone or in combination with other nucleic acids specific for the same target. Further, the term “biological sensor” specifically includes secondary biological sensors containing a consensus sequence derived from comparing two or more known biological sensors that bind to a given target.

In general, a minimum of approximately 3 nucleotides, preferably at least 5 nucleotides, are necessary to effect specific binding. The only apparent limitations on the binding specificity of the target/biological sensor complexes of the invention concern sufficient sequence to be distinctive in the binding biological sensor and sufficient binding capacity of the target substance to obtain the necessary interaction. Oligonucleotides of sequences shorter than 10 bases may be feasible if the appropriate interaction can be obtained in the context of the environment in which the target is placed. Although the biological sensors described herein are single-stranded or double-stranded, it is contemplated that biological sensors may sometimes assume triple-stranded or quadruple-stranded structures.

The specifically binding biological sensors need to contain the sequence that confers binding specificity, but may be extended with flanking regions and otherwise derivatized. In preferred embodiments of the invention, biological sensor binding sites will be flanked by known, amplifiable sequences, facilitating the amplification of the biological sensors by PCR or other amplification techniques. In a further embodiment, the flanking sequence may comprise a specific sequence that preferentially recognizes or binds a moiety to enhance the immobilization of the biological sensor to a substrate.

The biological sensors found to bind to the targets may be isolated, sequenced, and/or amplified or synthesized as conventional DNA or RNA molecules. Alternatively, biological sensors of interest may comprise modified oligomers. Any of the hydroxyl groups ordinarily present in biological sensors may be replaced by phosphonate groups, phosphate groups, protected by a standard protecting group, or activated to prepare additional linkages to other nucleotides, or may be conjugated to solid supports. The 5′ terminal OH is conventionally free but may be phosphorylated. Hydroxyl group substituents at the 3′ terminus may also be phosphorylated. The hydroxyls may be derivatized by standard protecting groups. One or more phosphodiester linkages may be replaced by alternative linking groups. These alternative linking groups include, exemplary embodiments wherein P(O)O is replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

The biological sensors used as starting materials in the process of the invention to determine specific binding sequences may be single-stranded, double-stranded or triple-stranded DNA or RNA. In a preferred embodiment, the sequences are single-stranded DNA. The use of DNA eliminates the need for conversion of RNA biological sensors to DNA by reverse transcriptase prior to PCR amplification. Furthermore, DNA is less susceptible to nuclease degradation than RNA. In preferred embodiments, the starting biological sensor will contain a randomized sequence portion, generally including from about 10 to 400 nucleotides, more preferably 20 to 100 nucleotides. The randomized sequence is flanked by primer sequences that permit the amplification of biological sensors found to bind to the analyte. The flanking sequences may also contain other convenient features, such as restriction sites. These primer hybridization regions generally contain 10 to 30, more preferably 15 to 25, and most preferably 18 to 20, bases of known sequence.

Both the randomized portion and the primer hybridization regions of the initial oligomer population are preferably constructed using conventional solid phase techniques. Such techniques are well known in the art, such methods being described, for example, in Froehler, et al., (1986a, 1986b, 1988, 1987). Biological sensors may also be synthesized using solution phase methods such as triester synthesis, known in the art. For synthesis of the randomized regions, mixtures of nucleotides at the positions where randomization is desired are added during synthesis.

Any degree of randomization may be employed. Some positions may be randomized by mixtures of only two or three bases rather than the conventional four. Randomized positions may alternate with those which have been specified. Indeed, it is helpful if some portions of the candidate randomized sequence are in fact known.

Biological sensors may be selected, synthesized or modified to exhibit multiple functional properties. Such multifunctional biological sensors may be produced, for example, by synthesis of a single nucleic acid, incorporating the sequences of two or more biological sensors of different function; by ligating or cross-linking two or more biological sensors of different function; or by adding a separate functional moiety to a biological sensor exhibiting binding specificity for a desired target analyte. Association of multiple biological sensors into a single multifunctional complex may occur by covalent interaction or non-covalent interaction, according to any methods known in the art for associating two or more nucleic acids.

In one embodiment, a biological sensor with binding affinity for a first target analyte may be prepared as described below. A conserved sequence, exhibiting the first binding activity, may be incorporated into a new library of biological sensors, each containing new randomized sequences. The new library may be screened for binding affinity to a second target analyte, resulting in the production of a single biological sensor with binding affinity for two different analytes.

Nucleic Acid Chips and Biological Sensor Arrays

Nucleic acid chips provide a means of rapidly screening analytes for their ability to hybridize to a potentially large number of single stranded biological sensor probes immobilized on a solid substrate. In preferred embodiments, the biological sensors are DNA. Specifically contemplated are chip-based DNA technologies such as those described by Hacia et al., 1996 and Shoemaker et al., 1996. These techniques involve quantitative methods for analyzing large numbers of samples rapidly and accurately. The technology capitalizes on the binding properties of single stranded DNA to screen samples. (Pease et al., 1994; Fodor et al., 1993; Southern et al., 1994; Travis, 1997; Lipshutz et al., 1995; Matson et al., 1995; each of which is incorporated herein by reference.)

A biological sensor chip or array consists of a solid substrate upon which an array of single stranded biological sensor molecules have been attached. For screening, the chip or array is contacted with a sample containing analyte which is allowed to bind. The degree of stringency of binding may be manipulated as desired by varying, for example, salt concentration, temperature, pH and detergent content of the medium. The chip or array is then scanned to determine which biological sensors have bound to the analyte. Prior to the present invention, DNA chips were typically used to bind to target DNA or RNA molecules in a sample.

A variety of DNA chip formats are described in the art, for example U.S. Pat. Nos. 5,861,242 and 5,578,832, incorporated herein by reference. The structure of a biological sensor chip or array comprises: (1) an excitation source; (2) an array of probes; (3) a sampling element; (4) a detector; and (5) a signal amplification/treatment system. A chip may also include a support for immobilizing the probe.

In particular embodiments, a biological sensor may be tagged or labeled with a substance that emits a detectable signal, such as an organic semiconductor. The tagged or labeled species may be fluorescent, phosphorescent, or luminescent, or it may emit Raman energy or it may absorb energy. When the biological sensor binds to a targeted analyte, a signal is generated that is detected by the chip. The signal may then be processed in several ways, depending on the nature of the signal.

The biological sensor may be immobilized onto an integrated microchip that also supports a phototransducer and related detection circuitry. Alternatively, a biological sensor may be immobilized onto a membrane or filter that is then attached to the microchip or to the detector surface itself.

The biological sensors may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, incorporated by reference. A variety of methods have been utilized to either permanently or removably attach the biological sensors to the substrate. Exemplary methods are described above under the section on immobilization. When immobilized onto a substrate, the biological sensors are stabilized and may be used repeatedly.

Exemplary substrates include nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane) and photopolymers which contain photoreactive species such as nitrenes, carbenes and ketyl radicals capable of forming covalent links with target molecules (U.S. Pat. Nos. 5,405,766 and 5,986,076, each incorporated herein by reference).

Binding of biological sensor to a selected support may be accomplished by any of several means. For example, DNA is commonly bound to glass by first silanizing the glass surface, then activating with carbodiimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellulose membranes, the DNA probes are spotted onto the membranes. A UV light source (Stratalinker, from Stratagene, La Jolla, Calif.) is used to irradiate DNA spots and induce cross-linking. An alternative method for cross-linking involves baking the spotted membranes at 80° C. for two hours in vacuum. Further, it is specifically contemplated that the biological sensor may be bound to an immobilized indicator species. Therefore, in a preferred embodiment of the invention, an organic semiconductor is immobilized to a solid substrate and the biological sensors attached to the immobilized organic semiconductor. Alternatively, the organic semiconductor/biological sensor complex may be bound via the organic semiconductor or the polynucleotide to the substrate.

Specific biological sensors may first be immobilized onto a membrane and then attached to a membrane in contact with a transducer detection surface. This method avoids binding the biological sensor onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

Bioactive Agents and Analytes

In certain embodiments, it may be desirable to couple specific bioactive agents to one or more biological sensors for delivery to a target. For example, a biological sensor that bound selectively or specifically to tumor cells could be prepared using the methods disclosed above, with the tumor cells as the target analyte. The purified biological sensor could then be used as a targeting moiety to direct the delivery of a tumoricidal agent. In other embodiments, a biological sensor could be used for targeted delivery of an agent that promotes cell growth, for example to facilitate wound healing. Alternatively, a biological sensor could be coupled to gene therapy vectors such as viral and non-viral vectors. The vectors may encode various bioactive agents, as discussed below. In still other embodiments, it may be desirable to produce high affinity biological sensors against analytes that may be bioactive agents. Such biological sensors may be of use, for example, for detection and quantification of a target analyte in a sample of human tissue or fluid.

Non-limiting examples of such agents include cytokines, chemokines, pro-apoptosis factors, pro-angiogenic factors and anti-angiogenic factors. The term “cytokine” is a generic term for proteins released by one cell population that act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor (FGF); prolactin; placental lactogen, OB protein; tumor necrosis factor(TNF)-α and TNF-β; mullerian-inhibiting substance; mouse gonadotropin-associated peptide; inhibin; activin; vascular endothelial growth factor (VEGF); TIE-2; endothelin-1 receptor (EDNRA); vascular permeability factor receptor (VEGFR1); basic fibroblast growth factor receptor (bFGFR); integrin; thrombopoietin (TPO); nerve growth factors such as NGF-β; platelet-growth factor; transforming growth factors (TGFs) such as TGF-α. and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-α, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M- CSF, EPO, kit-ligand or FLT-3, angiostatin, thrombospondin, endostatin, and LT. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-α, MIP1-β, and IP-10. The skilled artisan will recognize that certain cytokines are also known to have chemoattractant effects and could also be classified under the term chemokines.

Phage Display

In certain embodiments, it may be desirable to use random amino acid sequences in the form of a phage display library for use as target analytes. The phage display method has been used for a variety of purposes (see, for example, Scott and Smith, 1990, 1993; U.S. Pat. Nos. 5,565,332, 5,596,079, 6,031,071 and 6,068,829, each incorporated herein by reference.

Generally, a phage display library is prepared by first constructing a partially randomized library of cDNA sequences, encoding all possible amino acid combinations. The cDNA sequences are inserted in frame into, for example, a viral coat protein for a phage such as the fuse 5 vector (U.S. Pat. No. 6,068,829). The cDNAs are expressed as random amino acid sequences, incorporated into a coat protein such as the gene III protein of the fuse 5 vector. The randomized peptides are thus displayed on the external surface of the phage, where they can bind to biological sensors. Phage binding to the biological sensor may be separated from unbound phage using standard methods as disclosed above, for example by flow cytometry and cell sorting. If desired, it is possible to collect bound phage, detach them from the biological sensor by exposure to an appropriate solution and proceed with another round of binding and separation. This iterative process results in the selection of phage with an increased specificity for the biological sensor.

Once phage of an appropriate binding stringency have been obtained, it is possible to determine the amino acid sequence of the binding peptide by sequencing the portion of the phage genome containing the cDNA, for example by using PCR primers that flank the cDNA insertion site. Phage lacking any cDNA insert may be used as a control to ensure that binding is specific.

The skilled artisan will realize that phage display may be used to select for short (between 3 and 100, more preferably between 5 and 50, more preferably between 7 and 25 amino acid residues long) peptides that can bind to a desired biological sensor. Such peptides may be of use, for example, as potential inhibitors or activators of enzyme or protein function.

In Vivo Production of Biological Sensors

The lac operon regulates the transcription of DNA into mRNA for translation into β-galactosidase, permease, and transacetylase. These three enzymes are necessary for the bacteria to metabolize lactose. The expression of β-galactosidase in a variety of cells including E. coli has become an invaluable tool for marking transfection (the insertion of foreign genes) and expression of genes. By using a medium that contains a substrate (x-gal) for β-galactosidase that turns blue upon the action of the enzyme, one can detect the insertion of foreign genes into the β-galactosidase gene. In the absence of an insert into β-galactosidase, expression of the lac operon results in a blue color on x-gal, while the presence of an insert results in a white bacterial plaque.

The lac repressor gene within the lac operon encodes a protein that prevents the a enzymes in the lac operon from being expressed. The repressor protein is inactivated by binding to an inducer or de-repressor, resulting in expression of β-galactosidase and causing a blue color to form on x-gal. In the absence of an inducer or de-repressor, only the repressor is translated from the lac operon and no lactose (or color-producing substrate) metabolism occurs. The repressor gene is always translated first, before the enzymes in the operon. Therefore, if the transcription of the repressor gene is altered too much, the downstream genes will not be expressed (no blue color).

This method can be carried one step further. By inserting a marker gene in place of the (β-galactosidase gene, induction or derepression of the lac operon results in the expression of the new protein in place of β-galactosidase. Other markers used to replace galactosidase include green fluorescent protein (GFP), chloramphenicol acetyltransferase (CAT), luciferase, and nitrate reductase (U.S. Pat. No. 5,902,728). The GFP makes the cells fluoresce green, CAT converts radiolabeled chloramphenicol to a more soluble product that appears in a different place on a thin-layer chromatographic plate, luciferase produces bioluminescence in transfected cells, and nitrate reductase can produce calorimetric, fluorescent, or luminescent products in cells.

A mutagen assay based on the lac operon has been incorporated into cultured animal cells and whole transgenic animals (Big Blue™ mice and rats). Mutations in the repressor gene allow for unrestricted expression of β-galactosidase and production of blue colored substrate. Thus, mutagenic activity can be assayed by measuring the level of blue plaques obtained in the absence of induction. Further, by replacing the promoter of the lac operon with another promoter that is responsive to different regulatory factors, one can test for factors that bring about expression of any gene of choice, using marker gene expression.

The problem of using the above methods is that a specific promoter must be found for each agent (regulatory factor) that is to be detected. To do this, microbes that already have the appropriate metabolic machinery to detect the presence of a specific agent must be found or genetically engineered. This has been done at Oak Ridge National Laboratory for detection of explosives using genetically engineered pseudomonads. The presence of the specific agent (explosive) induces expression of a gene encoding GFP. Thus, the pseudomonad produces GFP when spread out over ground containing landmines (leaking explosives).

It would be much more convenient to genetically engineer the lac operon of a microbe like E. coli to detect a variety of agents (analytes). By using biological sensors that can be selected to bind to almost any desired target, this problem may be solved. DNA sequences comprising biological sensors may be incorporated into the repressor gene or its promoter in such a way that when the target analyte binds to the biological sensor, expression of the repressor protein is inhibited and β-galactosidase or another marker gene is expressed. Thus, blue colonies or other markers will appear in the presence of the designed inducer (i.e. the target analyte). Since biological sensors with high affinity against virtually any target analyte can be prepared and sequenced using the methods described herein, it would be possible to design an appropriate biosensor microorganism that is capable of detecting almost any molecule in the environment.

It is envisioned within the scope of the invention that the target analyte could bind to the biological sensor either within the intact repressor gene or its promoter, or in the mRNA transcript of the repressor gene, prior to its translation into protein. High-affinity binding of analyte to mRNA would interfere with ribosomal binding and mRNA translation. For this reason, in preferred embodiments it may be desirable for the biological sensor insertion site to be close to the ribosomal binding site of the repressor gene sequence, allowing for steric hindrance of ribosomal binding.

This process can be extended to a large library of biological sensors, each of which is inserted into the same site of the repressor gene or its promoter. The process can thus be used to select an appropriate biological sensor for a target analyte of choice by selecting for a bacterial clone that is colored blue or otherwise marked only in the presence of the target analyte. Amplification of the selected clone and DNA sequencing would result in the identification of biological sensor sequences that can bind with high affinity to the target analyte. The normal inducer will also work because it acts on the repressor gene product (the repressor protein itself) rather than the machinery to translate the gene into protein (like the biological sensor). This is an important positive control to confirm the fidelity of the system. This method would allow for screening of biological sensor libraries and selection and amplification of biological sensors with high affinity for a target analyte. Purified biological sensors of appropriate binding specificity may be obtained either by chemical synthesis or by PCR or other amplification processes using primers selected to flank the biological sensor insertion site.

The process may also be adapted for use with a recognition complex system. By cloning in E. coli (see U.S. Pat. No. 5,902,728, incorporated herein by reference) or another appropriate host that has been genetically engineered to produce the organic semiconductor, then growth on an appropriate medium will result in the production of biological sensors that are already operatively linked to the organic semiconductor.

The skilled artisan will realize that the methods and compositions described above are not limited to the lac operon expressed in E. coli. A variety of cells, expression vectors and marker genes may be used within the scope of the present invention, the only requirement being that an assay be available to detect binding of target analyte to biological sensor within the host cell.

In preferred embodiments, the marker genes used encode selectable marker proteins. Non-limiting examples of selectable markers include the herpes simplex virus thymidine kinase (Wigler et al., 1977), hypoxanthine-guanine phosphoribosyltransferase (Szybalska et al., 1962) and adenine phosphoribosyltransferase genes (Lowy et al., 1980), in tk-, hgprt- or aprt-cells, respectively. Also, antimetabolite resistance may be used as the basis of selection for dhfr, that confers resistance to methotrexate (Wigler et al., 1980; O'Hare et al., 1981); gpt, that confers resistance to mycophenolic acid (Mulligan et al., 1981); neo, that confers resistance to the aminoglycoside G-418 (Colberre-Garapin et al., 1981); hygro, that confers resistance to hygromycin (Santerre et al., 1984); and bar, that confers resistance to bialaphos and related herbicides (White et al., 1989; U.S. Pat. No. 5,489,520, incorporated herein by reference.)

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

DNA Based Recognition Complex System

Methods and Materials

All oligonucleotides were obtained from Ransom Hill Biosciences, Sigma Chemical Co., or Genosys Corp. The BACA1FI and BACA6RI gene probes were synthesized from published sequences (Reif et al., 1994) for portions of the capsular antigen gene of virulent strains of Bacillus anthracis. Precast 4-20% gradient and 10% homogenous polyacrylamide gels made with Tris-Borate-EDTA (TBE) buffer as well as DNA ladder (Amplisize; 50-2,000 bp) standards were run on a mini Protean II electrophoresis system (BioRad). DALM was biosynthesized in Escherichia coli strain JM109 bacteria and partially purified as described in Bruno et al., 1998. All polymerase chain reaction (PCR) reagents, including dideoxynucleotides, were from a “Silver Sequence” kit, and binding buffer (BB) was composed of 0.5M NaCl, 10 mM Tris-HCl, and 1 mM MgCl₂ in deionized water (pH 7.5 to 7.6; Bruno, 1997).

Two types of arrays of biological sensors were generated: 1) a naturally occurring overlapping random (N) 60 mer; and 2) a contiguous or ligated array. In the latter array, biological sensor diversity was increased, compared to the starting random 60 mers, by truncating longer chains with the addition of dideoxynuclotides during a PCR step and covalently linking non-contiguous DNA chains together with Taq DNA ligase.

The PCR chain termination step involved addition of 6.6 μg of random (N) 60 mer as a self-priming (due to partial hybridization) PCR template with 8 μl of each deoxy/dideoxynucleotide (ie., d/ddA, d/ddC, d/ddG, d/ddT) and 20 μl (80 units) of Taq polymerase per tube. The tubes were PCR amplified using the following temperature profile: 96° C. for 5 min, followed by 40 cycles of 96° C. for 1 min, 25° C. for 1 min, and 72° C. for 1 min. PCR extension was completed at 72° C. for 7 min and tubes were stored at 4 to 6° C. until electrophoresed. The collection of biological sensor species present as overlapping random (N) 60 mers or as ligated and truncated DNAs constituted a library of biological sensors.

For both types of DNA arrays, 3.3 μg (typically 5 to 10 μl) of library DNA was diluted with 2× loading buffer and loaded into each well of precast 10% or 4-20% gradient mini TBE polyacrylamide gels and electrophoresed in cold 1× TBE for 1 h at 100 V per gel. If DNA was to be visualized in the gel, gels were stained with 0.5 μg/ml ethidium bromide in TBE for 10 min, followed by rinsing in deionized water for 30 min and photography on a 300 nm ultraviolet transilluminator using Polaroid type 667 film.

Arrays of biological sensors were generated from library DNA separated by electrophoresis (size and charge). Analyte binding and nucleic acid hybridization to the biological sensor arrays were assayed as follows. Gels were cut into strips containing the one-dimensional DNA arrays of either type and were added to 10 ml of BB. Gel strips were allowed to equilibrate in their respective buffers for 10 min at room temperature (RT) with gentle shaking and were then scanned as described below prior to addition of analytes. All DNA analytes were added at a final concentration of 5 μg/ml and all protein analytes were added at a final concentration of 10 μg/ml in BB for 1 h at RT with gentle shaking. Gels were gently rinsed twice in 10 ml of BB, carefully repositioned and rescanned on a luminescence spectrometer.

To compare the fluorescence emission spectrum of DALM in the presence or absence of DNA, 50 μl drops of slow hardening epoxy resin were placed in black microtiter plate wells and overlaid with 50 μl of undiluted bacterial DALM. The DALM and epoxy were incubated in a covered plate for three to four days at ambient temperature. Excess DALM was removed by five washes with 200 μl of deionized water. All fluid was decanted and emission spectra were acquired before and after the addition of 50 μl (30 μg) of random 60 mer DNA.

A Perkin-Elmer (Beaconsfield, Buckinghamshire, UK) model LS 50B luminescence spectrometer equipped with a plate reader was used in the thin layer chromatography (TLC) plate mode to scan biological sensor arrays in gel slices before and after addition of various analytes. After minor swelling or shrinkage in each of the reaction buffers, gel strips were generally 95 to 96 mm in length, with the DNA array being contained in the lowermost 65 mm of each gel strip. Gel strips were scanned with an excitation of 260 nm (10 nm slits), emission of 420 nm (10 nm slits) and 1 mm resolution (i.e., scanned in 1 mm increments). In some cases, DALM and random 60 mer DNA were scanned separately and in combination using an excitation wavelength of 360 nm (excitation maximum for DALM).

An alternative method for attaching an array of recognition complexes to glass or other solid surfaces was developed. In this method, DALM was attached directly to a glass slide. Biological sensors can be attached to DALM using magnesium ion binding as shown in FIG. 3, or by covalent or other attachment techniques discussed above. Glass slides were cleaned with alcoholic potassium hydroxide, washed with DI (deionized water) and dried overnight. To approximately 150 ml of acetone was added 8 ml of water and 12 ml of 3-aminopropyltriethoxysilane. Acetone was added to a final volume of 200 ml. The slides were placed on the bottom of a rectangular plastic storage container and the acetone solution was poured over them. After two hours at room temperature on an orbital shaker (75 rpm) the slides were washed twice with acetone.

The presence of amino groups on the surface of the glass was examined. To 20 ml of saturated sodium borate, 5 ml of 5% w/v 2,4,6-trinitrobenzenesulfonate was added. Slides were placed in the solution and incubated at 37° C. for 2 hours, then rinsed with DI. The presence of amino groups was indicated by a yellow color.

DALM was covalently attached to the amino groups on the surface of the glass. Reduced synthetic DALM (55.2 mg) was dissolved in 2 to 3 ml of 0.1 M NaOH. 0.1 M MOPS buffer (pH 7) was added to a final volume of 50 ml. The DALM solution was poured over the glass slides in the storage container. Additional MOPS buffer was added until all slides were completely covered. EDC (N,N-(3-dimethylaminopropyl)-N′-ethyl-carbodiimide hydrochloride, 130 mg) was dissolved in MOPS buffer and immediately added to the slides, while shaking on an orbital shaker. This addition was repeated every 15 min for an additional four times. After another hour, 200 mg of EDC was added. The slides were incubated at room temperature for another two hours with shaking, then rinsed and dried overnight. DALM was covalently attached to the glass slides. Although glass was used in this example, the skilled artisan will realize that any solid surface capable of being coated with 3-aminopropyltriethoxysilane could be used in the practice of the invention.

Results

Gel electrophoresis of random DNA libraries showed that a high degree of partial hybridization occurs between members of the library, leading to an aggregated collection of hybrids that appear as a smear on electrophoretic gels (data not shown). The electrophoretic migration of the array varied slightly from lane to lane in the gel.

Fluorescence emission of the biological sensor arrays with or without bound analyte was scanned using a 260 nm excitation to compare baseline fluorescence of the empty TLC plate reader, random N60 mer DNA in a gradient polyacrylamide gel (scanned at a locus with high DNA concentration), and bacterial DALM.

Random DNA in a polyacrylamide gel excited at 260 nm returned most of its energy in the ultraviolet region of the spectrum (not shown). DALM excited at 260 nm yields extensive fluorescence in the blue region of the spectrum (not shown). Emission wavelengths in the visible region of DALM's emission spectrum that augment the minor visible DNA emission peaks are most desirable for detection of analyte binding. A less prominent emission peak (420 nm) was selected for further analysis. Use of this excitation wavelength also avoided the high background fluorescence from DALM and the TLC plate reader observed between 265 to 370 nm and 500 to 540 nm, respectively.

The fluorescence emission spectra of DALM (attached to an epoxy layer) before and after interaction with random 60 mer DNA was compared. Excitation was performed at 360 nm (excitation maximum of DALM). The fluorescence of DALM with and without added biological sensors indicated enhanced fluorescence intensity and an emission spectrum shift of DALM after binding DNA. This demonstrates a fluorescence energy transfer from DALM to bound DNA and possible fluorescence enhancement of analyte-DNA array interactions in embodiments where DALM is used. In preferred embodiments, DALM serves as a photonic-electronic transducer and conductor for an attached array biological sensor layer.

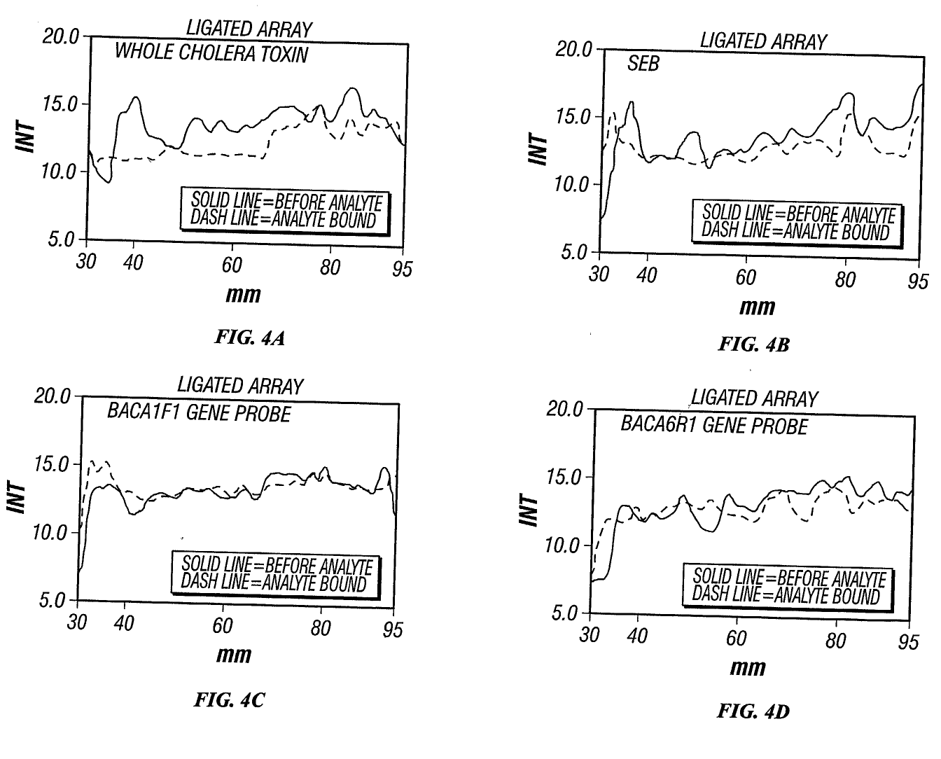

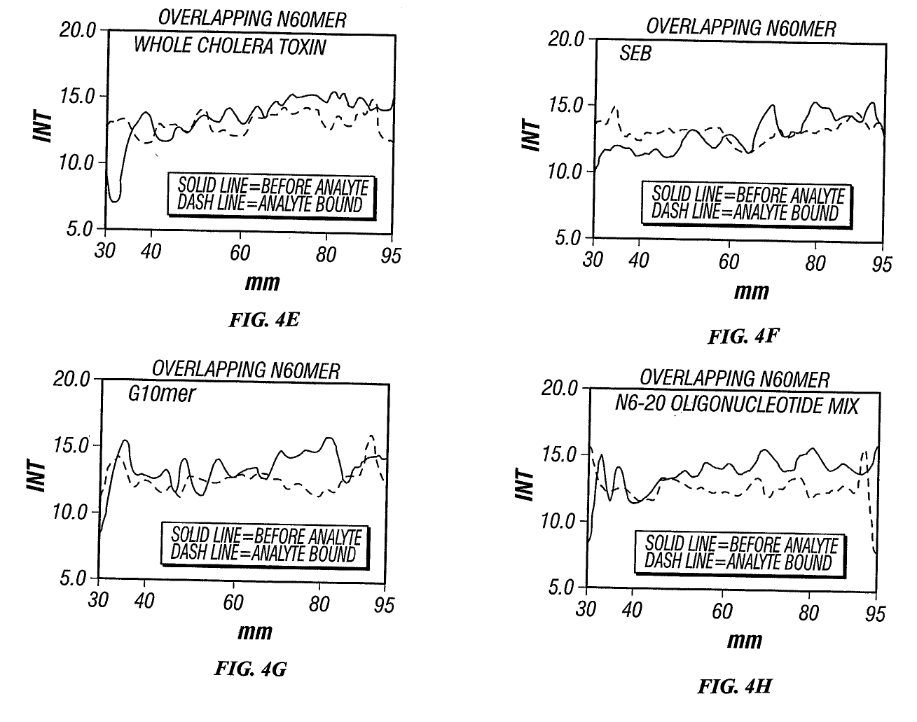

FIG. 4 shows a comparison of spatial fluorescence spectra for two different types of biological sensor arrays (ligated—FIGS. 4A-4D—versus random 60 mers—FIGS. 4E-4H) before and after addition of various analytes. The biological sensor arrays were electrophoresed in 10% polyacrylamide gels and fluorescence scanning was performed using an excitation of 260 nm and emission wavelength of 420 nm.

Spatial fluorescence scans of the different analyte interactions with two differently prepared biological sensor arrays suggested that the nature of the analyte and the type of array influenced the shape of the resultant scan (FIGS. 4A-4H). However, some common features (e.g., peaks and valleys) existed between related scans of each array taken before (solid line) and after (dashed line) analyte binding. Most of these shared features appear to be dampened upon interaction with the analyte (FIGS. 4A-4H), suggesting energy absorption by the DNA array-bound analyte. However, at specific wavelengths the fluorescence emission apparently increased upon binding of analyte (FIGS. 4A-4H).

It is apparent from FIG. 4 that the ligated array produced an emission spectrum different from the random 60 mer array when identical analytes were added (compare FIG. 4A vs. FIG. 4E and FIG. 4B vs. FIG. 4F). It is also apparent that for a given biological sensor array, binding to a different analyte resulted in a different (and apparently unique) fluorescence emission spectrum (compare whole cholera toxin, SEB, BACA1F1 gene probe and BACA6R1 gene probe). These results validate the concept of using a recognition complex array to generate unique photochemical signatures capable of identifying individual analytes.

### Example 2

Interaction of Recognition Complex System With Whole Cholera Toxin and DALM

Materials and Methods

Randomized 40 mer template DNA flanked by 5′ polyA and 3′ polyT (10 mer) regions was obtained from Genosys Corp. and PCR amplified in the presence of ddNTPs and 2 units of Taq ligase. Cholera toxin was obtained from Sigma Chemical Co. (St. Louis, Mo.). Ten μl of PCR product per gel lane was mixed 1:1 with DNA loading buffer and electrophoresed at 100V in 10% polyacrylamide precast minigels in TBE. Gels were then treated with bacterially synthesized DALM and/or cholera toxin in 1× binding buffer (BB). Gel lanes were cut and separated and scanned for fluorescence intensity at 260 nm excitation and 420 nm emission, using a Perkin-Elmer LS-50B spectrofluorometer and fiber optic plate reader attached in the TLC plate mode. The gel lanes were scanned before and after the addition of analyte (0.1 mg/ml of cholera toxin for 1 hr. at ambient temperature with mixing). DNA gels were 65 mm long and care was taken to place gels in precisely the same position before and after mixing with analyte.

Results

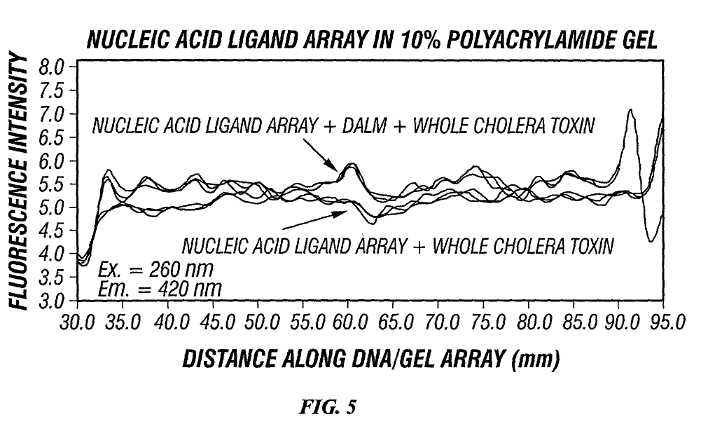

FIG. 5 illustrates differences in spatial fluorescence patterns for biological sensor arrays in 10% polyacrylamide gels with 0.1 mg/ml whole cholera toxin with and without DALM augmentation. Multiple (3 each) scans of the same DNA array in the presence and absence of analyte and/or DALM resulted in reproducible fluorescence emission profiles (FIG. 5). Addition of DALM primarily amplified the low-level fluorescence of the array DNA array and additionally changed the spatial fluorescence characteristics.

These results demonstrate the reproducibility of the photochemical signature resulting from analyte binding to an array of recognition complexes. It further demonstrates that recognition complex arrays, comprising biological sensors operatively linked to DALM, show enhanced fluorescence signals depending on the specific interaction between analyte and the individual biological sensor species.

### Example 3

Alternative Recognition Complex Array

Materials and Methods

Lyophilized random DNA oligonucleotides of 40-60 bases (50 O.D. units) in length were obtained from Ransom Hill Bioscience, Inc. (Ramona, Calif.) and rehydrated in 1 ml autoclaved deionized water. Random oligomers were placed on ice and allowed to anneal for >1 hr. prior to electrophoresis. Ten μl of undiluted DNA oligomers were loaded across the wells of 10-20% Tris-glycine gradient polyacrylamide minigels (BioRad, Hercules, Calif.) and electrophoresed in cold 1× TBE buffer at 200V, 150 mA, and 35 W max for 75 min. Polyacrylamide gels were removed and imprints of the gels were cut with one-half of DNA Bind™ (Corning-CoStar, NOS coated) microtiter plates in a procedure hereafter referred to as the molecular cookie cutter approach. This generated small circular plugs of gel containing spatially resolved regions of the electrophoresed random DNA molecules.

These gel plugs were cut out and placed into the appropriate wells of the microtiter plate to ensure a spatial replica of the original gel. The DNA in each plug was eluted out of the gel plug onto the DNA Bind™ plate and immobilized onto the plate surface by addition of a DNA hybridization buffer (HB, 200 μL per well) at 37° C. for 2 hr.

Results

Several biotinylated target DNAs were hybridized to immobilized DNA in a DNA Bind™ plate. Plates were washed three times in HB. Hybridization patterns of the biotinylated target DNAs were detected by addition of 1:500 streptavidin-peroxidase (Southern Biotechnologies, Birmingham, Ala.) in 2% bovine serum albumin (BSA)-HB solution for 30 min at room temperature (RT). Plates were washed three more times in BB and exposed to 200 μl tetramethyl benzidine (TMB; Kirkegaard Perry Laboratories, Gaithersburg, Md.) containing hydrogen peroxide for approximately 10 min at RT to visually detect hybridization patterns. The results (not shown) demonstrate that this is an alternative approach to generating a recognition complex array.

Strong adsorption of DALM to polystyrene microtiter wells was observed at low pH (pH 5.0 or lower). This non-covalent binding was stable in the neutral pH (7 to 7.5) range, but not in alkaline pH. It is contemplated that DALM may also be immobilized using N-oxy-succinimide (NOS) treated polystyrene surfaces. An alternative method would be to link a diamino-aliphatic chain such as 1,5-diaminopentane (cadaverine), 1,6-hexane, or poly-L-lysine to the NOS and then to DALM via a carbodiimide linkage with carboxyl groups in DALM.

### Example 4

Neutralization Of Biohazardous Agents Using DALM

In a preferred embodiment of the instant invention, biological sensors with high affinity for a target analyte are produced and purified using the disclosed methods. Such biological sensors may be used to neutralize biohazardous agents, such as viruses, microbes, spores or potentially single molecules.

High affinity biological sensors may be produced as disclosed in the preceding examples. Such biological sensors may be attached to a compound such as DALM. The biological sensor provides specificity of binding to the target. The DALM-biological sensor couplet is then used essentially as a photochemical transducer.

DALM is capable of absorbing electromagnetic radiation within a broad range of wavelengths and transmitting the absorbed energy to molecules or targets to which it is attached. DALM attached to a target via a bound biological sensor is irradiated with a pulse of electromagnetic radiation. The radiation may be transmitted in the form of visible light or infrared radiation, but other forms of irradiation, such as microwave, laser or radiofrequency are contemplated within the scope of the present invention. Iradiation results in absorption of energy by DALM, which is transmitted to the target. The resulting heating and production of reactive chemical species produces an explosive surface reaction that destroys the target.

DALM activated by hydrogen peroxide and bicarbonate and pulsed with microwave radiation acts as a photochemical transducer, releasing an intense pulse of visible light (not shown). High power pulsed microwave radiation (HPM), applied to solutions containing dissolved carbon dioxide (or bicarbonate), hydrogen peroxide and DALM generates sound, pulsed luminescence and electrical discharge. Microbes exposed to these conditions experience damage comparable to short time, high temperature insults, even though measurable localized temperatures were insufficient to cause the observed effects.

Materials and Methods

Bacillus anthracis spores were incubated with DALM and exposed to a high power microwave (HPM) pulse. Bacillus anthracis (BA; Sterne strain) spore vaccine (Thraxol™, Mobay Corp., Animal Health Division, Shawnee, Kans. 66201) was centrifuged, the supernatant decanted and the button washed with chilled deionized water. Dilute powdered milk solution was made to a concentration of 25 mg of powdered milk solids/ml of deionized water, filtered through a 0.2 micron filter. The BA button was resuspended in 1 ml of sterile milk solution to form a BA suspension.

For pulsed microwave exposure, 0.5 ml of BA spore suspension was placed into 0.2 micron-filter centrifuge tubes (Microfilterfuge™, Rainin Instrument Co., Inc., Woburn, Mass. 01888-4026). The spores were centrifuged onto the filter at 16,000×g for 15 min. The tubes were refilled with 1.5 ml of a reaction mixture consisting of 0.9 ml saturated sodium bicarbonate/luminol solution, 0.1 ml of 1:10 biosynthetic DALM, 0.6 ml of 1:10 diazoluminol, and 0.33 ml 3% hydrogen peroxide. All dilutions were made in saturated sodium bicarbonate/luminol solution. The final dilution of DALM was 1:1000. A detailed description of the reaction mixture has been published (Kiel et al., 1999a; Kiel et al., 199b).

The filter, with the BA spores, was inserted into the tube to a level just below the meniscus of the fluid. The solution was exposed to 10 pulses per second of HPM (1.25 GHz, 6 μs pulse, 2 MW peak incident power), starting at 3 minutes and 22 seconds after placing the reaction mixture in front of the microwave waveguide. The exposure lasted for 13 min and 28 sec. Total radiation exposure was for 48 msec. The temperature of the sample, continuously monitored with a non-perturbing, high-resistance temperature probe (Vitek™), began at 25.3° C. and reached an end point of 64° C., below the lethal temperature for anthrax spores.

Results

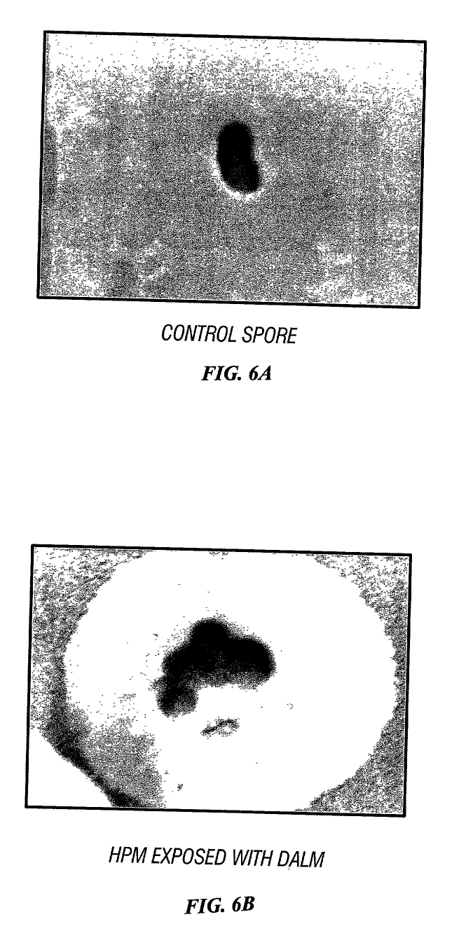

FIG. 6 shows the result of this procedure. The control spore on the left was exposed to HPM in the absence of DALM. It remained intact. The anthrax spore on the right of FIG. 6 was exposed to HPM in the presence of DALM. The spore lysed, with its contents spread around the remnants of the spore (FIG. 6). The effect of HPM and DALM on anthrax spores shows that DALM may be used to neutralize biohazardous agents against which high affinity biological sensors are prepared by the methods disclosed herein.

### Example 5

In Vivo Production of Biological Sensors and DALM

Methods

Starting materials for preparing biological sensors in vivo may be obtained by chemical synthesis of random nucleic acid sequences. The nucleic acids may be designed with appropriate restriction endonuclease sequences and amplification sequences incorporated into their 5′ and 3′ ends.

Plasmids or other expression vectors for screening may be digested with an appropriate restriction endonuclease and the precipitated with sodium acetate and ethanol at—20° C. and dried. The dried DNA is dissolved in water and 10× calf intestinal alkaline phosphatase reaction buffer and 2 Units of calf intestinal alkaline phosphates are added. The reactions are incubated at 37° C. for 15 min, 55° C. for 45 min and the enzyme heat denatured for 10 min at 75° C. The digests are extracted with equal volumes of phenol/chloroform (50:50) and the supernatant extracted again with an equal volume of chloroform. The resulting dephosphorylated DNA is ethanol precipitated in the presence of sodium acetate at −20° C. The dried DNA is dissolved in DNA buffer to 10 ng/μl .

If desired, the inserts may be subjected to PCR amplification by incubating at 96° C. for 2 min, followed by 35 cycles of 94° C. for 1 min; 60° C. for 1 min; and 72° C. for 1 min. Final chain elongation may be performed at 72° C. for 5 min. The amplification product is pooled and digested with EcoR1, then diluted to 10 ng/μl. The digested amplification product, containing the putative biological sensor, is ligated into the dephosphorylated plasmids.

Different concentrations of diluted putative biological sensor may be used to give varying ratios of biological sensor to vector. A ratio of 1:1 or 2:1 is preferred. Biological sensor, vector and water are combined to give a total volume of 6.5 μl. Each vector is set up without biological sensor DNA as a control. The samples are heated to 42° C. for 10 min. and cooled on ice. To each sample is added 1 μl of 5 mM ATP, 2 μl 5× ligation buffer and 0.5 μl T₄ DNA ligase. The samples are incubated at 16° C. overnight.

An overnight culture of E. coli JM 109 is set up in YT broth at 37° C., with shaking. On the following day an aliquot of this is diluted 1:50 with YT broth and is grown to 0.3-0.4 OD₅₅₀, Cells are spun at 8,000 rpm for 10 min at 4° C., supernatant removed and 8.0 ml ice-cold Competent A solution (10 mM sodium chloride, 50 mM manganese chloride, 10 mM sodium acetate) added. The resuspended cells are left on ice for 20 min, spun for 10 min at 8,000 rpm at 4° C. and resuspended in 1.0 ml of ice-cold Competent B solution (75 mM calcium chloride, 100 mM manganese chloride, 10 mM sodium acetate)

Cells are resuspended and 100 μl aliquots are pipetted into glass tubes on ice. Aliquots of ligation mixture containing 10 μg DNA are added to each tube. One tube has only cells and no DNA to check the viability of cells used. Tubes are incubated on ice for 30 min and then placed in a heating block at 42° C. to heat shock for 2 min. One ml of warmed YT broth is added to each tube and samples are incubated without shaking at 37° C. for 20 min.

Warmed YT ampicillin agar plates are plated with 100 μl aliquots of each sample and one YT agar plate is plated with an additional 100 μl aliquot from the tube with cells but no DNA. Plates are incubated at 37° C. overnight. Resulting colonies are tested by DNA mini preparation for the presence of biological sensor DNA.

This methodology can be used for expressing biological sensors in bacteria (E. coli) or eukaryotic cells (RAW mouse macrophages, EMT-6 mouse mammary carcinoma cells, HeLa human cervix carcinoma cells, etc.) The plasmids can also be used for the production of DALM if the 1.1 kb fragment of barley nitrate reductase gene is included (see U.S. Pat. No. 5,902,728, May 11 1999; and U.S. Pat. No. 5,464,768, Nov. 7, 1995; incorporated herein by reference). If protein or other analytes that bind to the biological sensors of interest are introduced into the transformed host cells (by electroporation, active transport, passive diffusion or any other transport mechanism), they can bind to the vector DNA or mRNA biological sensor sequences that have affinity for the target analyte. Binding of analyte to biological sensor inhibits expression of down stream genes for antibiotic resistance or nitrate reductase activity or inhibit translation of mRNA that includes the biological sensor sequences.

These changes provide for a selection process that can be used for in vivo screening of biological sensors. It has been reported (Kiel, Parker, Grubbs, and Alls, In Chemical and Biological Sensing, Proceedings of SPIE, vol. 4036, pp. 92-102) that it is possible to control the expression of DALM and the death of a recombinant E. coli by controlling its expression of the NR1.1 gene fragment. The binding of analyte to a biological sensor in these plasmids would inhibit the killing of the E. coli or growth stoppage of the eukaryotic cell under nitrating (nitrite production) conditions. By cloning a variety of biological sensor sequences, the one(s) that bind the analyte will be selected for by survival of the clone containing it.

Alternatively, colonies containing a biological sensor sequence with affinity for an analyte could be marked with colorimetric or fluorescent dyes for the expression or lack of expression of nitrate reductase, GFP, CAT, luciferase or other marker genes caused by the interaction of the ligand sequences with the analyte. Besides bacterial, mammalian, and human cell types, this approach could be adapted to yeast. Sarcchomyces lack nitrate reductases. Therefore, cloning of the gene could be easily detected.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Bruno and Yu, lmmunomagnetic-electrochemiluminescent detection of Bacillus anthracis spores in soil matrices. Appl. Environ. Microbiol. 62: 3474-76, 1996.

Bruno, In vitro selection of DNA to chloroaromatics using magnetic microbead-based affinity separation and fluorescence detection. Biochim. Biophys. Res. Comm. 234, 117-120, 1997.

Bruno et al., Preliminary electrochemiluminescence studies of metal ion-bacterial diazoluminomelanin (DALM) interactions. J. Biolumin. Chemilumin. 13: 117-123, 1998.

Colberre-Garapin et al., J. Mol. Biol., 150:1, 1981.

Effenhauser, et al Anal. Chem., 66:2949-2953, 1994.

Effenhauser, et al. Anal Chem., 65:2637-2642, 1993.

Egholm et al., Nature, 365:566, 1993.

Ellington and Szostak, In vitro selection of RNA molecules that bind specific ligands. Nature 346: 818-822, 1990.

Ellington and Szostak, Selection in vitro of single stranded DNA molecules that fold into specific ligand-binding structures. Nature 355: 850-52, 1992

Fodor et al., Multiplexed biochemical assays with biological chips. Nature 364, 555-556, 1993

Freifelder, Physical Biochemistry Applications to Biochemistry and Molecular Biology, 2nd ed. Wm. Freeman and Co., New York, N.Y., 1982.

Froehler, B., Tet Lett. 27:5575-5578, 1986a.

Froehler, B., et al., Nucleic Acids Research, 14:5399-5467, 1986b.

Froehler, B., et al. Nucleosides and Nucleotides, 6:287-291, 1987.

Froehler, B., et al. Nucleic Acids Research, 16:4831-4839, 1988.

Frohman, In: PCR™ Protocols: A Guide To Methods And Applications, Academic Press, N.Y., 1990.

Gatto-Menking et al., Sensitive detection of biotoxoids and bacterial spores using an immunomagnetic electrochemiluminescence sensor. Biosensors Bioelectronics 10: 501-507, 1995.

Guild, The calorimetric properties of the spectrum. Philosophical Transactions of the Royal Society, A, 230:149-187, 1931.

Guiot and Couvreur (eds), Polymeric Nanoparticles and Microspheres, CRC Press, pp. 97-103, 1986.

Hacia et al., Nature Genetics, 14:441-447, 1996

Harrison et al., Science, 261:895-897, 1993.

Holmstrom, K. et al., Anal. Biochem. 209:278-283, 1993.

Innis et al., PCR Protocols, Academic Press, Inc., San Diego Calif., 1990.

Jacobson, et al., Anal Chem., 66:1107-1113, 1994.

Jayasena, S. D., Aptamers: An Emerging Class of Molecules That Rival Antibodies in Diagnostics. Clin. Chem. 45: 1628-1650, 1999.

Kiel et al. “Luminescent radio frequency radiation dosimetry.” Bioelectromagnetics 20:46-51, 1999a.

Kiel et al., “Pulsed microwave induced light, sound, and electrical discharge enhanced by a biopolymer.” Bioelectromagnetics 20:216-223, 1999b.

Klug and Famulok, All you wanted to know about SELEX. Mol. Biol. Reports 20: 97-107, 1994.

Kornberg and Baker, DNA Replication, 2d Ed., (Freeman), San Francisco, 1992

Kwoh et al., Proc. Nat. Acad. Sci. USA, 86: 1173, 1989.

Kugler et al., “Photoelectron spectroscopy and quantum chemical modeling applied to polymer surfaces and interfaces in light-emitting devices.” Accounts of Chemical Research 32:225-234, 1999.

Lipshutz et al., Using oligonucleotide probe arrays to access genetic diversity. Biotechniques 19: 442-447, 1995.

Lorsch and Szostak, In vitro selection of nucleic acid sequences that bind small molecules. In: Combinatorial Libraries: Synthesis, Screening and Application Potential. (R. Cortese, ed.) Walter de Gruyter Publishing Co., New York, pp. 69-86, 1996.

Lowy et al., Cell, 22:817, 1980.

Manz, et al., ,J. Chromatogr., 593:253-258, 1992.

Matson et al., Biopolymer synthesis on polypropylene supports: oligonucleotide arrays. Anal.

Biochem. 224: 110-116, 1995.

Mulligan et al., Proc. Nat'l Acad. Sci. USA, 78:2072, 1981.

Newton, et al. Nucl. Acids Res. 21:1155-1162, 1993.

Ohara et al., Proc. Nat'l Acad. Sci. USA, 86:5673-5677, 1989.

O'Hare et al., Proc. Nat'l Acad. Sci. USA, 78:1527, 1981.

Pease et al., Light-generated oligonucleotide arrays for rapid DNA sequence analysis. Proc.

Natl. Acad. Sci. USA 91: 5022-26, 1994.

Rasmussen, et al., Anal. Biochem, 198:138-142, 1991.

Reif et al., Identification of capsule-forming Bacillus anthracis spores with the PCR and a novel dual-probe hybridization format. Appl. Environ. Microbiol. 60:1622-25, 1994.

Roitt et al., In: Immunology, pp. 5-6, Gower Medical Publishing Ltd., London, 1985.

Running. J. A. et al., BioTechniques8:276-277, 1990.

Sambrook et al., In: Molecular Cloning: A Laboratory Manual, 2d Ed., Cold Spring Harbor Laboratory Press, Cold Spring Harbor, N.Y., 1989.

Santerre et al., Gene, 30: 147-156, 1984.

Scheit, Nucleotide Analogs, John Wiley, New York, 1980

Shoemaker et al., Nature Genetics 14:450-456, 1996.

Southern et al., Arrays of complementary oligonucleotides for analyzing the hybridization behaviour of nucleic acids. Nucleic Acids Res. 22: 1368-73, 1994.

Travis, Chips ahoy: microchips covered with DNA emerge as powerful research tools. Science News 151: 144-45, 1997.

Tsuda et al., Anal Chem., 62:2149-2152, 1990.

Tuerk, Systematic evolution of ligands by exponential enrichment: RNA ligands to bacteriophage T4 DNA polymerase. Science249: 505-510, 1990.

Tuerk, In vitro evolution of functional nucleic acids: high-affinity RNA ligands of HIV-1 proteins. Gene137: 33-39, 1993.

Tuerk, Using the SELEX combinatorial chemistry process to find high affinity biological sensors to target molecules. Meth. Mol. Biol. 67: 219-30, 1997.

Walker et al., Proc. Natl. Acad. Sci. USA, 89:392-396 1992.

Weetall, H. W. in: Methods in Enzymology, K. Mosbach (ed.), 44:134-148, 140, 1976

White et al., Nucleic Acids Res. 18:1062, 1989.

Wigler et al., Proc. Nat'l Acad. Sci. USA, 77:3567, 1980.

Woolley and Mathies, Proc Natl Acad Sci U S A, 91:11348-52, 1994.

Wright, A re-determination of the trichromatic coefficients of the spectral colours. Transactions of the Optical Society, London, 30: 141-164, 1929.

Wu et al., Genomics, 4:560, 1989.

Yu and Bruno, Irnmunomagnetic-electrochemi-luminescent detection of Escherichia coli 0157 and Salmonella typhimurium in foods and environmental water samples. Appl. Environ. Microbiol. 62: 587-92, 1996

U.S. Pat. No. 3,652,761

U.S. Pat. No. 3,970,518

U.S. Pat. No. 3,933,997

U.S. Pat. No. 4,230,685

U.S. Pat. No. 4,267,234

U.S. Pat. No. 4,677,055

U.S. Pat. No. 4,683,195

U.S. Pat. No. 4,683,202

U.S. Pat. No. 4,695,393

U.S. Pat. No. 4,800,159

U.S. Pat. No. 4,883,750

U.S. Pat. No. 5,003,050

U.S. Pat. No. 5,270,163

U.S. Pat. No. 5,296,375

U.S. Pat. No. 5,304,487

U.S. Pat. No. 5,376,963

U.S. Pat. No. 5,405,766

U.S. Pat. No. 5,424,545

U.S. Pat. No. 5,446,543

U.S. Pat. No. 5,475,096

U.S. Pat. No. 5,489,520

U.S. Pat. No. 5,539,082

U.S. Pat. No. 5,567,588

U.S. Pat. No. 5,578,832

U.S. Pat. No. 5,580,737

U.S. Pat. No. 5,582,981

U.S. Pat. No. 5,595,877

U.S. Pat. No. 5,610,287

U.S. Pat. No. 5,637,459

U.S. Pat. No. 5,641,629

U.S. Pat. No. 5,645,897

U.S. Pat. No. 5,650,275

U.S. Pat. No. 5,670,637

U.S. Pat. No. 5,683,867

U.S. Pat. No. 5,696,249

U.S. Pat. No. 5,705,337

U.S. Pat. No. 5,705,629

U.S. Pat. No. 5,707,796

U.S. Pat. No. 5,714,331

U.S. Pat. No. 5,719,262

U.S. Pat. No. 5,723,289

U.S. Pat. No. 5,723,592

U.S. Pat. No. 5,736,336

U.S. Pat. No. 5,763,173

U.S. Pat. No. 5,763,177

U.S. Pat. No. 5,766,855

U.S. Pat. No. 5,773,571

U.S. Pat. No. 5,773,598

U.S. Pat. No. 5,786,461

U.S. Pat. No. 5,789,157

U.S. Pat. No. 5,817,785

U.S. Pat. No. 5,818,044

U.S. Pat. No. 5,837,832

U.S. Pat. No. 5,837,860

U.S. Pat. No. 5,843,653

U.S. Pat. No. 5,853,984

U.S. Pat. No. 5,856,108

U.S. Pat. No. 5,856,174

U.S. Pat. No. 5,861,242

U.S. Pat. No. 5,864,026

U.S. Pat. No. 5,867,265

U.S. Pat. No. 5,874,218

U.S. Pat. No. 5,891,625

U.S. Pat. No. 5,904,824

U.S. Pat. No. 5,908,845

U.S. Pat. No. 5,958,691

U.S. Pat. No. 5,972,721

U.S. Pat. No. 5,986,076

U.S. Pat. No. 5,989,823

U.S. Pat. No. 6,001,577

U.S. Pat. No. 6,028,311

U.S. Pat. No. 6,030,776

U.S. Pat. No. 6,043,909

U.S. Pat. No. 6,072,464

GB App. No. 2,202,328

EPO App. No. 266,032

EPO App. No. 320,308

EPO App. No. 329,822

PCT/EP/01219

PCT App. No. U.S. 87/00880

PCT App. No. U.S. 89/01025

PCT App. No. WO 88/10315

PCT App. No. WO 89/06700

PCT App. No. WO 90/07641

PCT App. No. WO 91/19813

PCT App. No. WO 92/20702

PCT App. No. WO 94/05414

PCT App. No. WO 99/31275

What is claimed is:

1. A method of screening for biological sensors that bind to a selected analyte comprising:
a) preparing one or more candidate biological sensors;
b) inserting each candidate biological sensor into an expression vector comprising a target gene, wherein the insertion site is located near the 5′ end of the transcribed portion of the target gene;
c) transforming the expression vectors containing inserts into an appropriate host cell line;
d) exposing the transformed host cells to the selected analyte; and
e) identifying host cell colonies that do not express the target gene protein.

2. The method of claim 1, wherein the target gene encodes a selectable marker protein or a screenable marker protein.

3. The method of claim 2, wherein the screenable marker protein is β-glucuronidase (GUS), chloramphenicol acetyltransferase (CAT), luciferase or green fluorescent protein (GFP).

4. The method of claim 2, wherein the target gene is thymidine kinase, hypoxanthine guanine phosphoribosyltransferase, adenine phosphoribosyltransferase, dihydrofolate reductase, gpt, neo, hygro or bar.

5. The method of claim 1, wherein the target gene is a regulatory gene, and wherein the regulatory gene controls the expression of a marker gene.

6. The method of claim 5, wherein the regulatory gene is the lac repressor gene.

7. The method of claim 1, wherein the host cell is a prokaryotic cell, a eukaryotic cell, or a plant cell.

8. The method of claim 7, wherein the host cell is E. coli.

9. The method of claim 7, wherein the prokaryotic cell or the plant cell does not have an intact cell wall.

10. The method of claim 1, wherein the host cell line is capable of manufacturing DALM.

11. The method of claim 1, further comprising growing up the identified host colonies and repeating steps (d) and (e) until a colony is obtained that contains a biological sensor that binds with high affinity to the analyte.

12. A biological sensor produced by the method of claim 1.

13. The biological sensor of claim 12, wherein the biological sensor is covalently attached to a therapeutic moiety.

14. The biological sensor of claim 13, wherein the therapeutic moiety is a cytokine, a chemotherapeutic agent, a radioisotope, a cytotoxic agent, an enzyme, a protein, an inhibitor or a poison.

15. The biological sensor of claim 12, wherein the biological sensor is synthesized, modified, selected or ligated to another biological sensor to provide a multifunctional biological sensor.

16. The biological sensor of claim 15, wherein the multiple functions are selected from the group consisting of binding to a first analyte, binding to a second analyte, catalytic activity, chemical reactivity, photoreactivity, facilitating uptake into a cell, localization into a subcellular compartment, inhibition of enzyme activity and activation of enzyme activity.

17. A method of screening for products of biological reactions comprising:
a) preparing one or more recognition complexes containing biological sensors that bind with high affinity to the product of a biological reaction;
b) exposing the one or more recognition complexes to a sample; and
c) detecting binding of the one or more recognition complexes to the product.

18. The method of claim 17, wherein the method is used for high through-put screening, medium through-put screening or low through-put screening.

19. The method of claim 17, wherein the product is a cell surface protein.

20. The method of claim 17, wherein the product is a product of an enzymatic reaction.

21. The method of claim 20, further comprising detecting the presence of an inhibitor of the enzymatic reaction by the absence of binding to the product.

22. The biological sensor of claim 12, wherein the biological sensor comprises one or more specified nucleic acid sequences.
